SEQUENCE LISTING (37 sequences)

SEQ ID NO: 1 - protein, 418 aa, Unknown
  Escherichia coli YjeH
MSGLKQELGLAQGIGLLSTSLLGTGVFAVPALAALVAGNNSLWAWPVLIILVFPIAIVFAILGRHYPSAGGVAHFVGMAFGSRLERVTGWLFLSVIPVGLPAALQIAAGFGQAMFGWHSWQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEKMAAAASLPKIVVQLFGVGALWIACVIGYLACFASLNIYIQSFARLVWSQAQHNPDHYLARLSSRHIPNNALNAVLGCCVVSTLVIHALEINLDALIIYANGIFIMIYLLCMLAGCKLLQGRYRLLAVVGGLLCVLLLAMVGWKSLYALIMLAGLWLLLPKRKTPENGITT

SEQ ID NO: 2 - DNA, 1257 bp, Unknown
  Escherichia coli yjeH
atgagtggactcaaacaagaactggggctggcccagggcattggcctgctatcgacgtcattattaggcactggcgtgtttgccgttcctgcgttagctgcgctggtagcgggcaataacagcctgtgggcgtggcccgttttgattatcttagtgttcccgattgcgattgtgtttgcgattctgggtcgccactatcccagcgcaggcggcgtcgcgcacttcgtcggtatggcgtttggttcgcggcttgagcgagtcaccggctggctgtttttatcggtcattcccgtgggtttgcctgccgcactacaaattgccgccgggttcggccaggcgatgtttggctggcatagctggcaactgttgttggcagaactcggtacgctggcgctggtgtggtatatcggtactcgcggtgccagttccagtgctaatctacaaaccgttattgccggacttatcgtcgcgctgattgtcgctatctggtgggcgggcgatatcaaacctgcgaatatcccctttccggcacctggtaatatcgaacttaccgggttatttgctgcgttatcagtgatgttctggtgttttgtcggtctggaggcatttgcccatctcgcctcggaatttaaaaatccagagcgtgattttcctcgtgctttgatgattggtctgctgctggcaggattagtctactggggctgtacggtagtcgtcttacacttcgacgcctatggtgaaaaaatggcggcggcagcatcgcttccaaaaattgtagtgcagttgttcggtgtaggagcgttatggattgcctgcgtgattggctatctggcctgctttgccagtctcaacatttatatacagagcttcgcccgcctggtctggtcgcaggcgcaacataatcctgaccactacctggcacgcctctcttctcgccatatcccgaataatgccctcaatgcggtgctcggctgctgtgtggtgagcactttggtgattcatgctttagagatcaatctggacgctcttattatttatgccaatggcatctttattatgatttatctgttatgcatgctggcaggctgtaaattattgcaaggacgttatcgactactggcggtggttggcgggctgttatgcgttctgttactggcaatggtcggctggaaaagtctctatgcgctgatcatgctggcggggttatggctgttgctgccaaaacgaaaaacgccggaaaatggcataaccacataa

SEQ ID NO: 3 - DNA, 267 bp, Artificial Sequence
  Tn2335
gtggcctacaccttcgaccacgtcgtcgcctggcgctggtgcaccaaagaagacgcctacaactacacccacctcttcgatcaactccaaccacccttaatcgtgaccaccgacggacaaaaaaggcgcactcaaagccatcaccacgacctggccgacaacgaaaatccaacgctgcctcgtccacgtcaaacgcaacgtccaaaaacacgtcaccctaagacccgtgctgagctcgccgaaaagcactccggggtctctccttga

SEQ ID NO: 4 - DNA, 39 bp, Artificial Sequence
  Tn_5 F
tgaattcgagctcggtaccccaccgacgcgcatctgcct

SEQ ID NO: 5 - DNA, 50 bp, Artificial Sequence
  Tn_5 R
ggtgtggtgactttcagcagttcccgggggggaggaggcatgtggtgttg

SEQ ID NO: 6 - DNA, 50 bp, Artificial Sequence
  Tn_3 F
caacaccacatgcctcctcccccccgggaactgctgaaagtcaccacacc

SEQ ID NO: 7 - DNA, 42 bp, Artificial Sequence
  Tn_3 R
gtcgactctagaggatccccctcccaaaccattgaggaatgg

SEQ ID NO: 8 - DNA, 318 bp, Artificial Sequence
  CJ7
agaaacatcccagcgctactaatagggagcgttgaccttccttccacggaccggtaatcggagtgcctaaaaccgcatgcggcttaggctccaagataggttctgcgcggccgggtaatgcatcttctttagcaacaagttgaggggtaggtgcaaataagaacgacatagaaatcgtctcctttctgtttttaatcaacatacaccaccacctaaaaattccccgaccagcaagttcacagtattcgggcacaatatcgttgccaaaatattgtttcggaatatcatgggatacgtacccaacgaaaggaaacactc

SEQ ID NO: 9 - DNA, 39 bp, Artificial Sequence
  CJ7_yjeH F
acaccacatgcctcctccccagaaacatcccagcgctac

SEQ ID NO: 10 - DNA, 43 bp, Artificial Sequence
  CJ7_yjeH R
agttcttgtttgagtccactcatagtgtttcctttcgttgggt

SEQ ID NO: 11 - DNA, 44 bp, Artificial Sequence
  yjeH F
acccaacgaaaggaaacactatgagtggactcaaacaagaactg

SEQ ID NO: 12 - DNA, 44 bp, Artificial Sequence
  yjeH R
gactttcagcagttcccgggttatgtggttatgccattttccgg

SEQ ID NO: 13 - DNA, 23 bp, Artificial Sequence
  F92N F
cggctggctgaatttatcggtca

SEQ ID NO: 14 - DNA, 23 bp, Artificial Sequence
  F92N R
tgaccgataaattcagccagccg

SEQ ID NO: 15 - DNA, 23 bp, Artificial Sequence
  F351L F
caatggcatccttattatgattt

SEQ ID NO: 16 - DNA, 23 bp, Artificial Sequence
  F351L R
aaatcataataaggatgccattg

SEQ ID NO: 17 - DNA, 1161 bp, Unknown
  Corynebacterium glutamicum metB
ttgtcttttgacccaaacacccagggtttctccactgcatcgattcacgctgggtatgagccagacgactactacggttcgattaacaccccaatctatgcctccaccaccttcgcgcagaacgctccaaacgaactgcgcaaaggctacgagtacacccgtgtgggcaaccccaccatcgtggcattagagcagaccgtcgcagcactcgaaggcgcaaagtatggccgcgcattctcctccggcatggctgcaaccgacatcctgttccgcatcatcctcaagccgggcgatcacatcgtcctcggcaacgatgcttacggcggaacctaccgcctgatcgacaccgtattcaccgcatggggcgtcgaatacaccgttgttgatacctccgtcgtggaagaggtcaaggcagcgatcaaggacaacaccaagctgatctgggtggaaaccccaaccaacccagcacttggcatcaccgacatcgaagcagtagcaaagctcaccgaaggcaccaacgccaagctggttgttgacaacaccttcgcatccccatacctgcagcagccactaaaactcggcgcacacgcagtcctgcactccaccaccaagtacatcggaggacactccgacgttgttggcggccttgtggttaccaacgaccaggaaatggacgaagaactgctgttcatgcagggcggcatcggaccgatcccatcagttttcgatgcatacctgaccgcccgtggcctcaagacccttgcagtgcgcatggatcgccactgcgacaacgcagaaaagatcgcggaattcctggactcccgcccagaggtctccaccgtgctctacccaggtctgaagaaccacccaggccacgaagtcgcagcgaagcagatgaagcgcttcggcggcatgatctccgtccgtttcgcaggcggcgaagaagcagctaagaagttctgtacctccaccaaactgatctgtctggccgagtccctcggtggcgtggaatccctcctggagcacccagcaaccatgacccaccagtcagctgccggctctcagctcgaggttccccgcgacctcgtgcgcatctccattggtattgaagacattgaagacctgctcgcagatgtcgagcaggccctcaataacctttag

SEQ ID NO: 18 - DNA, 24 bp, Artificial Sequence
  metB_N_del F
tctagacgcccgcatactggcttc

SEQ ID NO: 19 - DNA, 37 bp, Artificial Sequence
  metB_N_del R
cccatccactaaacttaaacagatgtgatcgcccggc

SEQ ID NO: 20 - DNA, 38 bp, Artificial Sequence
  metB_C_del F
tgtttaagtttagtggatggggaagaaccacccaggcc

SEQ ID NO: 21 - DNA, 23 bp, Artificial Sequence
  metB_C_del R
gtcgaccaatcgtccagagggcg

SEQ ID NO: 22 - DNA, 1314 bp, Artificial Sequence
  Corynebacterium glutamicum metY
atgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctag

SEQ ID NO: 23 - DNA, 24 bp, Artificial Sequence
  metY_N_del F
tctagaccatcctgcaccatttag

SEQ ID NO: 24 - DNA, 37 bp, Artificial Sequence
  metY_N_del R
cccatccactaaacttaaacacgctcctgccaggttc

SEQ ID NO: 25 - DNA, 39 bp, Artificial Sequence
  metY_C_del F
tgtttaagtttagtggatgggcttggtacgcaaccaagg

SEQ ID NO: 26 - DNA, 23 bp, Artificial Sequence
  metY_C_del R
gtcgacgattgctccggcttcgg

SEQ ID NO: 27 - DNA, 1266 bp, Unknown
  Corynebacterium glutamicum lysC
gtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttggcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcaccttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaa

SEQ ID NO: 28 - DNA, 29 bp, Artificial Sequence
  lysC_L377K_5 F
tcctctagagctgcgcagtgttgaatacg

SEQ ID NO: 29 - DNA, 23 bp, Artificial Sequence
  lysC_L377K_5 R
aggtggaaatcttttcgatgttc

SEQ ID NO: 30 - DNA, 23 bp, Artificial Sequence
  lysC_L377K_3 F
gaacatcgaaaagatttccacct

SEQ ID NO: 31 - DNA, 29 bp, Artificial Sequence
  lysC_L377K_3 R
gactctagagttcacctcagagacgatta

SEQ ID NO: 32 - DNA, 1134 bp, Artificial Sequence
  Corynebacterium glutamicum  metX
atgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaa

SEQ ID NO: 33 - DNA, 28 bp, Artificial Sequence
  metX F
ggatcccctcgttgttcacccagcaacc

SEQ ID NO: 34 - DNA, 30 bp, Artificial Sequence
  metX R
ggatcccaaagtcacaactacttatgttag

SEQ ID NO: 35 - protein, 418 aa, Artificial Sequence
  YjeH F92N
MSGLKQELGLAQGIGLLSTSLLGTGVFAVPALAALVAGNNSLWAWPVLIILVFPIAIVFAILGRHYPSAGGVAHFVGMAFGSRLERVTGWLNLSVIPVGLPAALQIAAGFGQAMFGWHSWQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEKMAAAASLPKIVVQLFGVGALWIACVIGYLACFASLNIYIQSFARLVWSQAQHNPDHYLARLSSRHIPNNALNAVLGCCVVSTLVIHALEINLDALIIYANGIFIMIYLLCMLAGCKLLQGRYRLLAVVGGLLCVLLLAMVGWKSLYALIMLAGLWLLLPKRKTPENGITT

SEQ ID NO: 36 - protein, 418 aa, Artificial Sequence
  YjeH F351L
MSGLKQELGLAQGIGLLSTSLLGTGVFAVPALAALVAGNNSLWAWPVLIILVFPIAIVFAILGRHYPSAGGVAHFVGMAFGSRLERVTGWLFLSVIPVGLPAALQIAAGFGQAMFGWHSWQLLLAELGTLALVWYIGTRGASSSANLQTVIAGLIVALIVAIWWAGDIKPANIPFPAPGNIELTGLFAALSVMFWCFVGLEAFAHLASEFKNPERDFPRALMIGLLLAGLVYWGCTVVVLHFDAYGEKMAAAASLPKIVVQLFGVGALWIACVIGYLACFASLNIYIQSFARLVWSQAQHNPDHYLARLSSRHIPNNALNAVLGCCVVSTLVIHALEINLDALIIYANGILIMIYLLCMLAGCKLLQGRYRLLAVVGGLLCVLLLAMVGWKSLYALIMLAGLWLLLPKRKTPENGITT

SEQ ID NO: 37 - DNA, 5803 bp, Artificial Sequence
  pDCM2
  misc_feature (2895)..(2895): n is a, c, g, or t
gttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttttatcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgaaaaaggttaggaatacggttagccatttgcctgcttttatatagttcantatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggcggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccg

(19) United States
(12) Patent Application Publication
(10) Pub. No.: US 2023/0340549 A1
(43) Pub. Date: Oct. 26, 2023

(54) VARIANT OF INNER MEMBRANE PROTEIN AND METHOD FOR PRODUCING TARGET PRODUCT BY USING SAME

(71) Applicant: CJ CHEILJEDANG CORPORATION, Seoul (KR)

(72) Inventors: Seo Yun Kim, Seoul (KR); Seung Hyun Cho, Seoul (KR); Jae Min Lee, Seoul (KR); Min Ji Baek, Seoul (KR)

(73) Assignee: CJ CHEILJEDANG CORPORATION, Seoul (KR)

(21) Appl. No.: 17/787,263

(22) PCT Filed: Dec. 18, 2020
(86) PCT No.: PCT/KR2020/018713

§ 371 Date: Jun. 17, 2022

(30) Foreign Application Priority Data
Dec. 20, 2019 (KR) 10-2019-0172055

(51) Int. Cl. C12P 13/06; C12P 13/12; C12N 15/77; C12N 9/10; C12N 9/12

(52) U.S. Cl. CPC C12P 13/06; C12P 13/12; C12N 15/77; C12N 9/1029; C12Y 203/01031; C12N 9/1085; C12Y 205/01049; C12N 9/1217; C12Y 207/02004; C12N 2800/101

(57) ABSTRACT

The present disclosure relates to a variant of YjeH, which is an inner membrane protein, a microorganism including the same, and a method for producing a target product using the same.

### TECHNICAL FIELD

The present disclosure relates to a variant of YjeH, which is an inner membrane protein, and a method for producing a target product using same.

### BACKGROUND ART

O-Acetylhomoserine acts as a precursor of methionine, which is one of the essential amino acids in the human body. Homoserine, as a precursor of both methionine and threonine, is known to be converted into threonine via phosphohomoserine and converted into methionine via acetylhomoserine.

Methionine is used not only as an additive for feeds and foods but also as a raw ingredient for infusion solutions and pharmaceutical drugs and is produced by chemical and biological synthesis. A two-step process for producing L-methionine from an L-methionine precursor, which was produced by fermentation, via enzymatic conversion was published (International Publication No. WO 2008/013432).

In the two-step process, O-succinylhomoserine and O-acetylhomoserine are used as methionine precursors, and it is very important to produce O-acetylhomoserine with high yields for the purpose of economical mass production.

### DISCLOSURE

### Technical Problem

As a result of intensive efforts to increase production of O-acetylhomoserine or homoserine, the present inventors have found a variant of an inner membrane protein having enhanced O-acetylhomoserine or homoserine exporting activity, thereby completing the present disclosure.

### Technical Solution

The present disclosure provides a variant of YjeH, which is an inner membrane protein, including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1.

The present disclosure provides a polynucleotide encoding the variant.

The present disclosure provides a vector including the polynucleotide.

The present disclosure provides a microorganism including at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The present disclosure provides a method for producing a target product, the method including culturing a microorganism in a culture medium, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The present disclosure provides a method for producing methionine, the method including: producing O-acetylhomoserine by culturing a microorganism in a culture medium; and converting the O-acetylhomoserine into methionine by reacting the O-acetylhomoserine with a sulfide, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The present disclosure provides a method for producing glufosinate, the method including: producing O-acetylhomoserine or homoserine by culturing a microorganism in a culture medium; and converting the O-acetylhomoserine or homoserine into glufosinate, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The present disclosure provides a composition for producing homoserine including the microorganism or a culture of the microorganism.

The present disclosure provides a composition for producing O-acetylhomoserine including the microorganism or a culture of the microorganism.

The present disclosure provides a use of the variant of an inner membrane protein YjeH for producing O-acetylhomoserine or homoserine.

The present disclosure provides a use of the microorganism for producing O-acetylhomoserine or homoserine.

### Advantageous Effects

O-Acetylhomoserine or homoserine may be produced with higher yields by culturing a microorganism having the ability to produce O-acetylhomoserine or homoserine modified using the variant of YjeH, which is an inner membrane protein, according to the present disclosure compared to the non-modified proteins.

### BRIEF DESCRIPTION OF DRAWINGS

FIG. 1 is a schematic view of pDCM2 plasmid.

### BEST MODE

The present disclosure will be described in detail. Meanwhile, each description and embodiment disclosed in the present disclosure may be applied to different descriptions and embodiments herein. In other words, all combinations of various components disclosed in the present disclosure are included within the scope of the present disclosure. Furthermore, the scope of the present disclosure should not be limited by the descriptions provided below.

An aspect of the present disclosure to achieve the above-described objects provides a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1.

The variant of an inner membrane protein YjeH may have the ability to export O-acetylhomoserine or homoserine, i.e., O-acetylhomoserine or homoserine exporting activity.

As used herein, the term “homoserine” is an α-amino acid having a hydroxyl group in a side chain. Homoserine, as an intermediate of threonine and methionine biosynthesis in microorganisms and plants, is known to be produced from aspartic acid-4-semialdehyde and have a chemical formula of C₄H₉NO₃. Homoserine may be converted into O-acetylhomoserine by homoserine acetyl transferase in the presence of acetyl-CoA.

As used herein, the term “O-acetylhomoserine” refers to an acetyl derivative of L-homoserine, which is a specific intermediate material in the biosynthetic pathway of methionine in microorganisms. O-Acetylhomoserine is known to be produced by a reaction between homoserine and acetyl-CoA catalyzed by homoserine acetyltransferase. It has a chemical formula of C₆H₁₁NO₄. O-Acetylhomoserine may also be named O-acetyl-L-homoserine.

Specifically, in the variant of the protein according to the present disclosure, i) an amino acid corresponding to the 92^{nd} position may be substituted with a different amino acid, ii) an amino acid corresponding to the 351^{st} position may be substituted with a different amino acid, or iii) both of the amino acids corresponding to 92^{nd} position and 351^{st} position may be substituted with different amino acids, in the amino acid sequence of SEQ ID NO: 1, without being limited thereto. Substitution of the amino acids may be i) substitution of an amino acid corresponding to the 92^{nd} position with asparagine, or ii) substitution of an amino acid corresponding to the 351^{st} position with leucine, and more specifically, the variant of the protein may be a variant of the inner membrane protein YjeH in which i) the amino acid corresponding to the 92^{nd} position is substituted with asparagine, ii) the amino acid corresponding to the 351^{st} position is substituted with leucine, or iii) the amino acid corresponding to the 92^{nd} position is substituted with asparagine and the amino acid corresponding to the 351^{st} position is substituted with leucine, in the amino acid sequence of SEQ ID NO: 1, without being limited thereto.

As used herein, the term “inner membrane protein” refers to a protein as a member of the Amino Acid Efflux (AAE) Family within the Amino Acid-Polyamine-Organocation (APC) superfamily of transporters, mediating export of O-acetylhomoserine and/or homoserine. The protein is predicted to contain 12 transmembrane a helices, 10 of which form an inverted repeat fold that is characteristic of the APC superfamily. The inner membrane protein may be, for example, a protein including an amino acid sequence of SEQ ID NO: 1. The protein including the amino acid sequence of SEQ ID NO: 1 may be used interchangeably with protein having the amino acid sequence of SEQ ID NO: 1 or protein consisting of the amino acid sequence of SEQ ID NO: 1. In the present disclosure, the inner membrane protein may be used interchangeably with O-acetylhomoserine- or homoserine-exporting protein, protein having the ability to export O-acetylhomoserine or homoserine, protein having the O-acetylhomoserine or homoserine exporting activity, YjeH protein, or YjeH.

In the present disclosure, SEQ ID NO: 1 may refer to an amino acid sequence of YjeH, which is an inner membrane protein. Specifically, the SEQ ID NO: 1 may be an amino acid sequence of an inner membrane protein encoded by yjeH gene and having the O-acetylhomoserine or homoserine exporting activity. The amino acid sequence of SEQ ID NO: 1 may be obtained from a known database of the NCBI GenBank. For example, the amino acid sequence may be derived from Escherichia coli (E. coli), but is not limited thereto, and any sequence having the activity identical to that of the amino acid sequence may be included without limitation. In addition, although the inner membrane protein is defined as a protein including an amino acid sequence of SEQ ID NO: 1 in the present disclosure, the inner membrane protein does not exclude a mutation that may occur naturally or by addition of a meaningless sequence upstream or downstream of the amino acid sequence of the SEQ ID NO: 1, or a silent mutation thereof. As long as the protein has activity identical or equivalent to that of the protein comprising the amino acid sequence of SEQ ID NO: 1, it obviously belongs to the inner membrane protein of the present disclosure. For example, the inner membrane protein of the present disclosure may be a protein consisting of the amino acid sequence of SEQ ID NO: 1 or an amino acid sequence having at least 80%, 85%, 90%, 95%, 96%, 97%, 98%, 99%, 99.5%, or 99.7% homology or identity therewith. Also, it will be obvious that any protein having the amino acid sequence including deletion, modification, substitution, or addition in part of the amino acid sequence is within the scope of the present disclosure as long as the amino acid sequence retains the above-described homology or identity and effects equivalent to those of the protein.

That is, although the expressions “protein or polypeptide having an amino acid sequence set forth in a predetermined SEQ ID NO” and “protein or polypeptide including an amino acid sequence set forth in a predetermined SEQ ID NO” are used in the present disclosure, it is obvious that any protein having the amino acid sequence including deletion, modification, substitution, or addition in part of the amino acid sequence may also be used in the present disclosure, as long as the protein has the activity identical or equivalent to the polypeptide consisting of the predetermined amino acid sequence. For example, it is obvious that a “protein including the amino acid sequence of SEQ ID NO: 1” or “polypeptide including the amino acid sequence of SEQ ID NO: 1” belongs to a “protein consisting of the amino acid sequence of SEQ ID NO: 1” or “polypeptide consisting of the amino acid sequence of SEQ ID NO: 1” as long as the former has the same or equivalent activity to the latter.

As used herein, the term “variant” refers to a protein obtained by conservative substitution and/or modification of at least one amino acid different from that of the recited sequence while retaining functions or properties thereof. The variant may have an amino acid sequence different from the identified sequence due to substitution, deletion, or addition of several amino acids. Such variants may generally be identified by modifying at least one amino acid of the above amino acid sequence of the protein and evaluating properties of the modified protein. That is, the ability of the variant may be enhanced, may not be changed, or may be reduced relative to native protein. In addition, some variants may include variants from which at least one portion such as an N-terminal leader sequence or a transmembrane domain has been removed. Other variants may include variants in which a portion has been removed from the N- and/or C-terminus of a mature protein. The term “variant” may also be used interchangeably with other terms such as modification, modified protein, modified polypeptide, mutant, mutein, and divergent, and any terms used to indicate variation may also be used without limitation. In view of the objects of the present disclosure, the ability of the variant may be enhanced relative to a native or non-modified protein, without being limited thereto.

As used herein, the term “conservative substitution” refers to a substitution of one amino acid with another amino acid having a similar structural and/or chemical property. For example, the variant may include at least one conservative substitution while retaining at least one biological activity. Such amino acid substitution may generally occur based on similarity of polarity, charge, solubility, hydrophobicity, hydrophilicity, and/or amphipathic nature of a residue. For example, electrically charged amino acids with side chains include positively charged (basic) amino acids such as arginine, lysine, and histidine and negatively charged (acidic) amino acids such as glutamic acid and aspartic acid; and uncharged amino acids with side chains include nonpolar amino acids such as glycine, alanine, valine, leucine, isoleucine, methionine, phenylalanine, tryptophan, and proline, and polar or hydrophilic amino acids such as serine, threonine, cysteine, tyrosine, asparagine, and glutamine. Among the non-polar amino acids, aromatic amino acids include phenylalanine and tryptophan.

Variants may also include deletion or addition of amino acids that have minimal influence on properties and a secondary structure of a polypeptide. For example, a polypeptide may be conjugated to a signal (or leader) sequence at the N-terminus of a protein which co-translationally or post-translationally directs transfer of the protein. The polypeptide may also be conjugated with another sequence or linker to identify, purify, or synthesize the polypeptide.

In the present disclosure, the “substituted with a different amino acid” is not particularly limited as long as the amino acid after substitution is different from the amino acid before substitution.

In the present disclosure, the “substituted with a different amino acid” is not particularly limited as long as the amino acid after substitution is different from the amino acid before substitution. Specifically, the amino acid may be substituted with any one of lysine, histidine, glutamic acid, aspartic acid, glycine, alanine, valine, leucine, isoleucine, methionine, phenylalanine, tryptophan, proline, serine, threonine, cysteine, tyrosine, asparagine, and glutamine different from the previous amino acid sequence.

Specifically, in the amino acid sequence of SEQ ID NO: 1, phenylalanine, which is an amino acid corresponding to the 92^{nd} position, may be substituted with asparagine, or phenylalanine, which is an amino acid corresponding to the 351^{st} position, may be substituted with leucine. Meanwhile, in the present disclosure, it is obvious that the expression “substituted with a certain amino acid” means that the amino acid after substitution is different from the amino acid before substitution unless the expression “substituted with a different amino acid” is used.

Also, the substituted amino acid residue may include not only a natural amino acid, but also a non-natural amino acid. The non-natural amino acid may be, for example, a D-amino acid, (homo)-amino acid, (beta-homo)-amino acid, N-methyl amino acid, α-methyl amino acid, and uncommon amino acid (e.g., citrulline or naphthylalanine), but is not limited thereto. Meanwhile, in the present disclosure, it is obvious that the expression “substituted with a certain amino acid” means that the amino acid after substitution is different from the amino acid before substitution unless the expression “substituted with a different amino acid” is used.

The variant may be one in which at least any one of the amino acids corresponding to the 92^{nd} position and the 351^{st} position is substituted with an amino acid different from that before substitution in the amino acid sequence of SEQ ID NO: 1, or substituted with an uncharged amino acid with a side chain different from that before substitution, but is not limited thereto.

Specifically, the variant may be a variant of the protein in which i) the amino acid corresponding to the 92^{nd} position is substituted with a different amino acid, ii) the amino acid corresponding to the 351^{st} position is substituted with a different amino acid, or iii) the amino acids corresponding to the 92^{nd} position and the 351^{st} position are substituted with different amino acids, in the amino acid sequence of SEQ ID NO: 1, without being limited thereto. The substitution of amino acids may be i) substitution of the 92^{nd} amino acid with asparagine, or ii) substitution of the 351^{st} amino acid with leucine. More specifically, the variant may be a variant of the inner membrane protein YjeH in which i) the 92^{nd} amino acid is substituted with asparagine, ii) the 351^{st} amino acid is substituted with leucine, or iii) the 92^{nd} amino acid is substituted with asparagine and the 351^{st} amino acid is substituted with leucine, in the amino acid sequence of SEQ ID NO: 1, without being limited thereto.

As used herein, the term “corresponding to” refers to an amino acid residue at a position cited in a protein or polypeptide or an amino acid residue similar, identical, or homologous to the residue cited in the protein or polypeptide. As used herein, the term “corresponding region” generally refers to a region similar thereto in a related protein or a reference protein.

In the present disclosure, a particular numbering may be used for a position of an amino acid residue of the protein used in the present disclosure. For example, a position corresponding to an amino acid residue of the protein of the present disclosure may be renumbered by aligning an amino acid sequence of a protein to be compared and the amino acid sequence of the protein of the present disclosure.

The variant of an inner membrane protein YjeH provided in the present disclosure may refer to a variant having enhanced ability to export O-acetylhomoserine or homoserine compared to non-modified protein by substituting an amino acid at a specific position in the above-described inner membrane protein having the O-acetylhomoserine or homoserine exporting ability.

The variant of an inner membrane protein YjeH including i) substitution of the amino acid corresponding to the 92^{nd} position with asparagine, ii) substitution of the amino acid corresponding to the 351^{st} position with leucine, or substitution of both i) and ii), in amino acid sequence of SEQ ID NO: 1 may include any one of the amino acid sequences of SEQ ID NOS: 35 and 36, specifically consisting essentially of any one of the amino acid sequences of SEQ ID NOS: 35 and 36, more specifically consisting of any one of the amino acid sequences of SEQ ID NOS: 35 and 36, without being limited thereto.

In addition, the variant may include an amion acid sequence of any one of SEQ ID NS: 35 and 36, or an amino acid sequence in which at least any one amino acid of the 92^{nd} and 351^{st} amino acids is fixed in the amino acid sequences of SEQ ID NOS: 35 and 36 and having at least 80% homology with the amino acid sequences of SEQ ID NOS: 35 and 36, without being limited thereto. Specifically, variant of the present disclosure may include a polypeptide having at least 80%, 90%, 95%, 96%, 97%, 98%, or 99% homology or identity with any one of the amino acid sequences of SEQ ID NOS: 35 and 36. Also, it is obvious that any protein having the amino acid sequence including deletion, modification, substitution, or addition in part of the amino acid sequence at a position other than the 92^{nd} position and the 351^{st} position is within the a scope of the present disclosure as long as the protein retains the above-described homology or identity and effects equivalent to those of the variant.

As used herein, the term “homology” or “identity” refers to a degree of relevance between two given amino acid sequences or base sequences and may be expressed as a percentage. These terms homology and identity may often be used interchangeably.

Sequence homology or identity of conserved polynucleotides or polypeptides may be determined by standard alignment algorithm, and default gap penalties established by a program may be used together therewith. Substantially, homologous or identical sequences may hybridize with each other at least about 50%, 60%, 70%, 80%, or 90% of the entire sequence or the entire length under moderate or highly stringent conditions. In hybridized polynucleotides, polynucleotides including a degenerate codon instead of a codon may also be considered.

The sequence homology, similarity, or identity between two given polypeptides or polynucleotides may be determined using any known computer algorism such as “FASTA” program by using default parameters as introduced by, for example, Pearson et al. (1988) Proc. Natl. Acad. Sci. USA 85:2444. Alternatively, Needleman-Wunsch algorithm (1970, J. Mol. Biol. 48:443-453) performed in a Needleman program of The European Molecular Biology Open Software Suite (EMBOSS) package (Rice et al., 2000, Trends Genet. 16:276-277) (version 5.0.0 or later) may be used to determine the same (including GCG program package (Devereux, J. et al., Nucleic Acids Research 12:387 (1984)), BLASTP, BLASTN, FASTA (Atschul, S. F. et al., J MOLEC BIOL 215:403 (1990); Guide to Huge Computers, Martin J. Bishop, ed., Academic Press, San Diego, 1994, and CARILLO et al. (1988) SIAM J Applied Math 48:1073). For example, the homology, similarity, or identity may be determined using BLAST, from The National Center for Biotechnology Information database, or ClustalW.

The homology, similarity, or identity between polynucleotides or polypeptides may be determined, for example, by comparing sequence information using a GAP computer program, such as a program introduced by Needleman et al. (1970), J Mol Biol. 48:443, for example, as disclosed in Smith and Waterman, Adv. Appl. Math (1981) 2:482. In brief, the GAP program defines similarity as the number of aligned symbols (i.e., nucleotides or amino acids) which are similar, divided by the total number of symbols in a shorter of two sequences. Default parameters for the GAP program may include: (1) a binary comparison matrix (containing a value of 1 for identity and 0 for non-identity) a weighted comparison matrix of Gribskov et al. (1986) Nucl. Acids Res. 14:6745 disclosed in Schwartz and Dayhoff, eds., Atlas Of Protein Sequence And Structure, National Biomedical Research Foundation, pp. 353-358 (1979) (or EDNAFULL (EMBOSS version of NCBI NUC4.4) substitution matrix); (2) a penalty of 3.0 for each gap and an additional 0.10 penalty for each symbol in each gap (or gap open penalty of 10, and a gap extension penalty of 0.5); and (3) no penalty for end gaps.

Also, the sequence homology, similarity, or identity between two given polynucleotides or polypeptides may be identified by comparing sequences thereof by southern hybridization under defined stringent conditions, and the defined stringent hybridization conditions are within the scope of the technology and may be defined by a method well known to one of ordinary skill in the art (For example, J. Sambrook et al., Molecular Cloning, A Laboratory Manual, 2^{nd} Edition, Cold Spring Harbor Laboratory press, Cold Spring Harbor, New York, 1989; F. M. Ausubel et al., Current Protocols in Molecular Biology, John Wiley & Sons, Inc., New York).

As used herein, the term “variant of an inner membrane protein” or “variant of an inner membrane protein YjeH” may have the ability to export O-acetylhomoserine or homoserine and may be used interchangeably with a modified polypeptide having the ability to produce O-acetylhomoserine or homoserine, O-acetylhomoserine- or homoserine-producing modified polypeptide, modified polypeptide having the O-acetylhomoserine or homoserine producing ability, modified polypeptide having the O-acetylhomoserine or homoserine exporting activity, variant with the O-acetylhomoserine or homoserine exporting activity, O-acetylhomoserine- or homoserine-exporting variant, modified homoserine-exporting protein, modified O-acetylhomoserine-exporting protein, modified YjeH, variant YjeH, YjeH variant, YjeH mutant, or the like.

In addition, the protein may be derived from stains belonging to the genus Escherichia, the genus Erwinia, the genus Serratia, the genus Providencia, the genus Corynebacterium, the genus Pseudomonas, the genus Leptospira, the genus Salmonella, the genus Brevibacterium, the genus Hypomononas, the genus Chromobacterium, and the genus Norcardia, or fungi or yeast, specifically derived from strains belonging to the genus Escherichia, the genus Corynebacterium, and the genus Leptospira, and yeast, and more specifically derived from strains belonging to the genus Escherichia, e.g., Escherichia coli (E. coli), without being limited thereto.

The variant of an inner membrane protein YjeH may include mutation at the 92^{nd} position and/or the 351^{st} position in the amino acid sequence of SEQ ID NO: 1 and any variant including addition or deletion of an amino acid to or from the amino acid sequence of SEQ ID NO: 1 may be within the scope of the present disclosure as long as the amino acid corresponding to the 92^{nd} position and/or the 351^{st} position from the N-terminus of SEQ ID NO: 1 is substituted. The variant of an inner membrane protein YjeH may include an amino acid sequence including substitution of the 92^{nd} amino acid and/or 351^{st} amino acid with a different amino acid in the amino acid sequence of SEQ ID NO: 1 and may have enhanced activity compared to the inner membrane protein including the amino acid sequence of SEQ ID NO: 1 or not including mutation. The variant of an inner membrane protein YjeH may have a homology or identity of 80%, 85%, 90%, 95%, 96%, 97%, 98%, 99%, 99.5%, 99.7%, or more and less than 100% with SEQ ID NO: 1.

Mutation of the 92^{nd} or 351^{st} amino acid may be substitution of the 92^{nd} amino acid with asparagine or substitution of the 351^{st} amino acid with leucine.

Specifically, the variant of an inner membrane protein YjeH may include i) substitution of the 92^{nd} amino acid with asparagine, ii) substitution of the 351^{st} amino acid with leucine, or substitution of both i) and ii), in the amino acid sequence of SEQ ID NO: 1 and may have enhanced activity compared to the protein including the amino acid sequence of SEQ ID NO: 1 or the inner membrane protein derived from a wild-type microorganism before modification.

Another aspect of the present disclosure provides a polynucleotide encoding the variant of an inner membrane protein YjeH.

The inner membrane protein YjeH and the variant thereof are as described above.

As used herein, the term “polynucleotide” refers to a DNA or RNA strand having a certain minimum length or more as a polymer of nucleotides in which nucleotide monomers are linked to each other in the form of a long chain by covalent bonds, more specifically the polynucleotide may be a polynucleotide fragment encoding the variant.

The polynucleotide may include any nucleotide sequence encoding the variant of an inner membrane protein YjeH of the present disclosure without limitation. In the present disclosure, a gene encoding the amino acid sequence of the inner membrane protein YjeH is yjeH gene, and the gene may be derived from Escherichia coli (E. coli), without being limited thereto.

Specifically, the polynucleotide of the present disclosure may include various modifications made in a coding region provided not to change an amino acid sequence of a polypeptide expressed from the coding region by codon degeneracy or in consideration of codons preferred by a living organism in which the protein is expressed. Specifically, any polynucleotide sequence encoding the variant of an inner membrane protein YjeH in which the 92^{nd} or 351^{st} amino acid is substituted with a different amino acid in the amino acid sequence of SEQ ID NO: 1 may be included without limitation.

In addition, any sequence encoding an inner membrane protein, in which the 92^{nd} or 351^{st} amino acid of the amino acid sequence of SEQ ID NO: 1 is substituted with a different amino acid by hybridizing with a probe that may be prepared from known gene sequences, for example, sequences complementary to all or part of the nucleotide sequence under stringent conditions, may be included without limitation. The term “stringent conditions” refers to conditions which permit specific hybridization between polynucleotides. Such conditions are disclosed in detail in known documents (e.g., J. Sambrook et al.). For example, the conditions may include conditions for performing hybridization between genes having a high homology or identity, e.g., a homology or identity of 40% or more, specifically 90% or more, more specifically 95% or more, even more specifically 97% or more, and most specifically 99% or more, without performing hybridization between genes having a homology lower than the above homologies or identities, or performing hybridization once, specifically two or three times, under conventional washing conditions for Southern hybridization at a salt concentration and temperature of 60° C., 1×SSC, and 0.1% SDS, specifically 60° C., 0.1×SSC, 0.1% SDS, and more specifically 68° C., 0.1×SSC, and 0.1% SDS.

Hybridization requires that two polynucleotides have complementary sequences, although bases may mismatch according to the degree of stringency of hybridization. The term “complementary” is used to describe the relationship between nucleotide bases capable of hybridizing with each other. For example, with respect to DNA, adenine is complementary to thymine, and cytosine is complementary to guanine. Thus, the present disclosure may include not only a substantially similar nucleic acid sequence but also a nucleic acid fragment isolated but complementary to the entire sequence.

Specifically, the polynucleotides having homology or identity may be detected using the above-described hybridization conditions and using a hybridization process at a Tₘ value of 55° C. Also, the Tₘ value may be, but is not limited to, 60° C., 63° C., or 65° C., and may be appropriately adjusted by those skilled in the art according to the intended purposes.

An appropriate degree of stringency for hybridization of polynucleotides may depend on lengths of the polynucleotides and a degree of complementarity and parameters thereof are well known in the art (Sambrook et al., supra, 9.50-9.51, 11.7-11.8).

Another aspect of the present disclosure provides a vector including a polynucleotide encoding the variant of an inner membrane protein YjeH.

The inner membrane protein YjeH, the variant thereof, and the polynucleotide are as described above.

The vector of the present disclosure may be a DNA construct containing a nucleotide sequence of a polynucleotide encoding a target polypeptide and operably linked to a suitable expression regulatory region (expression regulatory sequence) so as to be able to express the target polypeptide in a suitable host cell. The expression regulatory sequence may include a promoter capable of initiating transcription, any operator sequence for regulating the transcription, a sequence encoding a suitable mRNA ribosome binding site, and a sequence for regulating termination of transcription and translation. When a suitable host cell is transformed with the vector, the vector may replicate or function independently from the host genome, or may integrate into genome thereof.

As used herein, the term “expression” includes any process related to producing a polypeptide such as transcription, post-transcriptional modification, translation, post-translational modification, and secretion, etc. without being limited thereto.

As used herein, the term “expression vector” refers to a linear or circular nucleic acid molecule including a coding sequence and an operably linked regulatory sequence for expression thereof.

As used herein, the term “operably linked” refers to a placing of a regulatory sequence to an appropriate position to regulate expression a coding sequence. Thus, “operably linked” includes a binding between a regulatory region of a functional domain having a known or desired activity such as a promoter, a stop codon, a signal sequence, or an enhancer and a target (gene or polypeptide) to regulate expression, secretion, or function of the target in accordance with the known or desired activity.

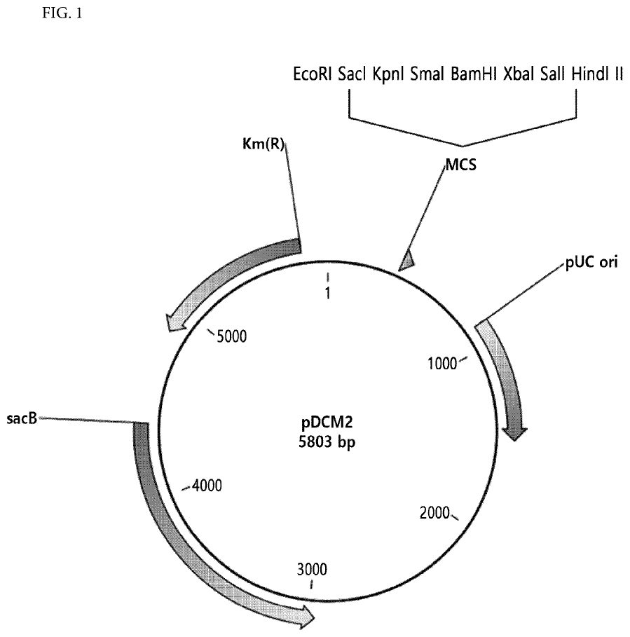

The vector used in the present disclosure is not particularly limited, and any known vector may be used. Examples of the known vectors may include natural or recombinant plasmids, cosmids, viruses, and bacteriophages. For example, pWE15, M13, MBL3, MBL4, IXII, ASHII, APII, t10, t11, Charon4A, and Charon21A, etc. may be used as a phage vector or cosmid vector, and pBR-based, pUC-based, pBluescriptII-based, pGEM-based, pTZ-based, pCL-based, and pET-based vectors may be used as a plasmid vector. Specifically, pDCM2, pACYC177, pACYC184, pCL, pECCG117, pUC19, pBR322, pMW118, and pCC1BAC vectors may be used. More specifically, the vector used in the present disclosure may be pDCM2 (FIG. 1, SEQ ID NO: 37) constructed for insertion and substitution of a gene in the chromosome of Corynebacterium, but is not limited thereto, and any known expression vector may be used.

For example, a polynucleotide encoding a target polypeptide may be inserted into a chromosome using a vector for chromosomal insertion into cells. The insertion of the polynucleotide into the chromosome may be performed by any method known in the art, for example, homologous recombination, without being limited thereto. The polynucleotide may further include a selection marker to confirm the chromosomal insertion. The selection marker is used to select cells that are transformed with the vector, that is, to confirm insertion of a desired nucleic acid molecule, and examples of the selection marker may include markers providing selectable phenotypes, such as drug tolerance, nutrient requirement, resistance to cytotoxic agents, or expression of surface polypeptide. Only cells expressing the selection marker are able to survive or to show different phenotypes under the environment treated with a selective agent, and thus the transformed cells may be selected.

Another aspect of the present disclosure provides a microorganism including at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The SEQ ID NO: 1, the inner membrane protein YjeH, the variant thereof, the polynucleotide encoding the variant, and the vector including the polynucleotide are as described above.

As used herein, the term, “microorganism including the variant of an inner membrane protein YjeH” refers to a microorganism with enhanced ability to export O-acetylhomoserine or homoserine prepared by introducing the variant of an inner membrane protein YjeH into a microorganism having weak ability to export O-acetylhomoserine or homoserine. Specifically, the microorganism is a microorganism expressing the variant of an inner membrane protein YjeH including at least one amino acid modification in the amino acid sequence of SEQ ID NO: 1, and the amino acid modification may include substitution of the 92^{nd} or 351^{st} amino acid from the N-terminus with a different amino acid.

In view of the objects of the present disclosure, the extracellular export of O-acetylhomoserine or homoserine of the microorganism including at least any one of the variant of an inner membrane protein YjeH; the polynucleotide encoding the variant; and the vector including the polynucleotide increases compared to wild-type or non-modified microorganisms, and thus the microorganism is characterized in that the ability to producing O-acetylhomoserine or homoserine is enhanced. While wild-type or non-modified microorganisms cannot export or can produce trace amounts of O-acetylhomoserine or homoserine, the ability of producing O-acetylhomoserine or homoserine may be enhanced according to the present disclosure by increasing the export amounts of O-acetylhomoserine or homoserine by introducing at least any one of the variant of an inner membrane protein YjeH; the polynucleotide encoding the variant; and the vector including the polynucleotide.

As used herein, the term “non-modified microorganism” does not exclude strains including mutation that may occur naturally in microorganisms and refers to a wild-type strain or a microorganism not including the variant of an inner membrane protein YjeH. The “non-modified microorganism” may be used interchangeably with “strain before modification”, “microorganism before modification”, “non-mutated strain”, “non-mutated microorganism”, “non-modified strain”, or “reference microorganism”.

As used herein, the term a protein “to be expressed/expressed” means a state in which a target protein is introduced into or modified to be expressed in a microorganism. In the case where the target protein is present in the microorganism, the activity of the protein may be enhanced compared to the activity of endogenous protein thereof or that before modification. In view of the objects of the present disclosure, the “target protein” may be the above-described variant of an inner membrane protein YjeH.

Specifically, the term “introduction of a protein” refers to providing activity of a particular protein to a microorganism which does not possess the protein or enhancing the activity of the protein compared to the intrinsic activity of the protein or the activity before modification. For example, the introduction of a protein may refer to introduction of a polynucleotide encoding the particular protein into chromosome or introduction of a vector including the polynucleotide encoding the particular protein into a microorganism, thereby expressing the activity of the protein. In addition, the “enhancement of activity” may mean that the activity of a particular protein of a microorganism is enhanced when compared with the intrinsic activity or the activity before modification. The term “intrinsic activity” may refer to activity of a particular protein possessed by a parent strain before transformation when a microorganism is transformed by natural or artificial genetic variation.

Specifically, the enhancement of the activity of the present disclosure may be achieved by way of at least one method selected from the group consisting of: a method of increasing the number of copies of a gene encoding the variant protein; a method of introducing mutation into an expression regulatory sequence of a gene encoding the variant protein; a method of replacing the expression regulatory sequence of the gene with a sequence encoding the variant protein and having stronger activity; a method of replacing a gene encoding a wild-type inner membrane protein on the chromosome with a gene encoding the variant protein; a method of additionally introducing mutation into a gene encoding the variant protein to enhance the activity of the variant protein; and a method of introducing the variant protein into the microorganism, without being limited thereto.

In the above description, the increasing the number of copies of the gene, although not particularly limited thereto, may be performed in a state operably linked to a vector, or by being inserted into the chromosome within a host cell. Specifically, the increase in gene copy number may be performed by introducing a vector, to which a polynucleotide encoding the variant protein of the present disclosure is operably linked, and which may replicate and function irrespective of a host cell, into a cell of the host. Alternatively, a vector, to which the polynucleotide is operably linked, capable of inserting the polynucleotide into the chromosome of the host cell, is introduced into the host cell. The insertion of the polynucleotide into the chromosome may be performed using a known method in the art, for example, by homologous recombination.

The modification of the expression regulatory sequence to increase expression level of the polynucleotide may be performed by, but is not particularly limited to, inducing mutation on the sequence by deletion, insertion, non-conservative substitution, conservative substitution, or any combination thereof to further enhance the activity of the expression regulatory sequence or replacement with a sequence having stronger activity. The expression regulatory sequence may include, but is not limited to, a promoter, an operator sequence, a sequence encoding a ribosome binding site, and a sequence regulating termination of transcription and translation.

A stronger promoter than the intrinsic promoter may be linked upstream of the polynucleotide expression unit, without being limited thereto. Examples of the stronger promoter known in the art may include CJ1 to CJ7 promoters (U.S. Pat. No. 7,662,943 B2), lac promoter, trp promoter, trc promoter, tac promoter, Lambda phage PR promoter, PL promoter, tet promoter, gapA promoter, SPL7 promoter, SPL13 (sm3) promoter (U.S. Ser. No. 10/584,338 B2), 02 promoter (U.S. Ser. No. 10/273,491 B2), tkt promoter, and yccA promoter, without being limited thereto.

In addition, the modification of the polynucleotide sequence on the chromosome may be performed by inducing mutation in the expression regulatory sequence by deletion, insertion, non-conservative substitution, conservative substitution, or any combination thereof to further enhance the activity of the polynucleotide sequence, or by replacing the nucleotide sequence with a nucleotide sequence modified to have a stronger activity, without being limited thereto.

The introduction of the protein and enhancement of the activity may increase the activity or concentration of the protein from the activity or concentration of a wild-type or non-modified microorganism strain, without being limited thereto.

In the present disclosure, the microorganism including the variant of an inner membrane protein YjeH may be a recombinant microorganism prepared by transformation using a vector including a polynucleotide encoding the variant of an inner membrane protein YjeH, but is not limited thereto.

As used herein, the term “transformation” refers to a process of introducing a vector including a polynucleotide encoding a target protein into a host cell in such a way that the protein encoded by the polynucleotide is expressed in the host cell. The transformed polynucleotide may be either in a form inserted into the chromosome of the host cell or in a form located outside the chromosome as long as the protein is expressed in the host cell. In addition, the polynucleotide includes DNA and/or RNA encoding the target protein. The polynucleotide may be introduced into the host cell in any form as long as the polynucleotide is introduced into the host cell and the protein is expressed therein. For example, the polynucleotide may be introduced into the host cell in the form of an expression cassette that is a gene construct including all of the essential elements required for self-expression. The expression cassette may generally include a promoter, a transcription termination signal, a ribosome binding site, and a translation termination signal which are operably linked to the polynucleotide. The expression cassette may be in the form of a self-replicable expression vector. Also, the polynucleotide may be introduced into the host cell in its original form and operably linked to a sequence required for the expression in the host cell, without being limited thereto.

Types of the microorganism having the homoserine producing ability are not particularly limited as long the microorganism is able to produce O-acetylhomoserine or homoserine, and specifically the microorganism may be any microorganism belonging the genus Corynebacterium, the genus Escherichia, the genus Enterobacter, the genus Erwinia, the genus Serratia, the genus Providencia, and the genus Brevibacterium, more specifically a microorganism belonging to the genus Corynebacterium.

More specifically, the microorganism belonging to the genus Corynebacterium may be Corynebacterium glutamicum, Corynebacterium ammoniagenes, Corynebacterium crudilactis, Corynebacterium deserti, Corynebacterium efficiens, Corynebacterium callunae, Corynebacterium stationis, Corynebacterium singulare, Corynebacterium halotolerans, Corynebacterium striatum, Corynebacterium pollutisoli, Cxorynebacterium imitans, Corynebacterium testudinoris, or Corynebacterium flavescens, and any microorganisms belonging to the genus Corynebacterium may be used without limitation.

In the present disclosure, a parent strain of the microorganism may be a microorganism in which a gene weakening the biosynthesis pathway of O-acetylhomoserine or homoserine is additionally inactivated or the biosynthesis pathway of O-acetylhomoserine or homoserine is enhanced to increase production of O-acetylhomoserine or homoserine, without being limited thereto.

Specifically, to inactivate the gene weakening the biosynthesis pathway of O-acetylhomoserine or homoserine, for example, expression of metB gene (SEQ ID NO: 17) encoding cystathionine gamma-synthase involved in a degradation pathway of O-acetylhomoserine or metY gene (SEQ ID NO: 22) encoding O-acetylhomoserine (thiol)-lyase involved in a degradation pathway of O-acetylhomoserine may be weakened or inactivated.

To enhancing the biosynthesis pathway of O-acetylhomoserine or homoserine, for example, genetic mutation may be introduced into IysC gene (SEQ ID NO: 27) encoding aspartokinase, or expression of metX gene (SEQ ID NO: 32) encoding O-acetylhomoserine transferase may be amplified.

However, the embodiment is not limited thereto, the ability to produce O-acetylhomoserine or homoserine may be enhanced by any known methods for gene expression regulation.

As used herein, the term “enhancement/increase” is a concept including an increase in the activity compared to the intrinsic activity.

The enhancement or increase of gene activity may be achieved by application of various methods well known in the art. Examples of the methods may include at least any one selected from the group consisting of: a method of increasing the copy number of a gene; a method of introducing mutation into an expression regulatory sequence of the gene; a method of replacing the expression regulatory sequence of the gene with a sequence having stronger activity; a method of additionally introducing mutation into the gene in order to enhance the activity of the gene; a method of introducing a foreign gene into a microorganism; and any combination thereof, without being limited thereto.

As used herein, the term “inactivation” of a protein means that the activity of the protein is weakened compared to the intrinsic activity, or that the protein has no activity.

The inactivation of the protein may be achieved by way of various methods well known in the art. Examples of the methods may include: a method of deleting a part or the entirety of a gene encoding the protein on the chromosome including removing the activity of the protein; a method of replacing the gene encoding the protein on the chromosome with a mutated gene to reduce the enzymatic activity; a method of introducing mutation into an expression regulatory sequence of a gene encoding the protein on the chromosome; a method of replacing the expression regulatory sequence of the gene encoding the protein with a sequence having a weak activity or no activity (e.g., a method of replacing a promoter of the gene with a promoter weaker than the endogenous promoter); a method of deleting a part or the entirety of the gene encoding the protein on the chromosome; a method of introducing an antisense oligonucleotide (e.g., antisense RNA), which inhibits translation from an mRNA into the protein via a complementary binding to a transcript of the gene on the chromosome; a method of making the attachment of a ribosome impossible by forming a secondary structure by artificially adding a complementary sequence to the Shine-Dalgarno (SD) sequence on the upstream of the SD sequence of the gene encoding the protein; and a reverse transcription engineering (RTE) method, which adds a promoter for reverse transcription to the 3′ terminal of the open reading frame (ORF) of the corresponding sequence, and a combination thereof, but are not particularly limited thereto.

Another aspect of the present disclosure provides a method for producing a target product including culturing a microorganism in a culture medium, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The SEQ ID NO: 1, the inner membrane protein YjeH, the variant thereof, the polynucleotide encoding the variant, the vector including the polynucleotide, and the microorganism including the same are as described above.

The target product may be O-acetylhomoserine or homoserine, but is not limited thereto.

As used herein, the term “culturing” refers to growing the microorganism in an appropriately adjusted environment. A culture process of the present disclosure may be performed according to an appropriate medium and culturing conditions known in the art. The culture process may be easily adjusted for use by a skilled person in the art according to a strain to be selected. The culturing of the microorganism may be performed in a batch process, a continuous process, a fed-batch process, etc., without being limited thereto.

As used herein, the term “culture medium” refers to a substance in which nutrients required for culturing the microorganism are mixed as main ingredients and supplies nutrients and growth factors as well as water which are essential for survival and growth. Specifically, although culture media and other culturing conditions for culturing the microorganism of the present disclosure are not particularly limited as long as the culture media are commonly used in culturing microorganisms, the microorganism of the present disclosure may be cultured in an ordinary medium containing appropriate carbon sources, nitrogen sources, phosphorus sources, inorganic compounds, amino acids, and/or vitamins under aerobic conditions while adjusting temperature, pH, and the like.

In the present disclosure, as the carbon sources, carbohydrates such as glucose, fructose, sucrose, and maltose; sugar alcohols such as mannitol and sorbitol; organic acids such as pyruvic acid, lactic acid, and citric acid; and amino acids such as glutamic acid, methionine, and lysine may be used. In addition, natural organic nutrients such as starch hydrolysates, molasses, blackstrap molasses, rice bran, cassava, sugar cane bagasse, and corn steep liquor may be used, and specifically carbohydrates such as glucose and sterile pretreated molasses (i.e., molasses converted to reduced sugars) may be used, and suitable amounts of any other carbon sources may also be used without limitation. These carbon sources may be used alone or in a combination of at least two thereof.

As the nitrogen sources, inorganic nitrogen sources such as ammonia, ammonium sulfate, ammonium chloride, ammonium acetate, ammonium phosphate, ammonium carbonate, and ammonium nitrate; and organic nitrogen sources such as amino acids, e.g., glutamic acid, methionine, and glutamine, peptone, NZ-amine, meat extract, yeast extract, malt extract, corn steep liquor, casein hydrolysate, fish or degradation products thereof, and defatted soybean cake or degradation products thereof may be used. These nitrogen sources may be used alone or in a combination of at least two thereof, without being limited thereto.

As the phosphorus sources, monopotassium phosphate, dipotassium phosphate, or sodium-containing salts corresponding thereto may be used. As inorganic compounds, sodium chloride, calcium chloride, iron chloride, magnesium sulfate, iron sulfate, manganese sulfate, calcium carbonate, and the like may be used. Also, amino acids, vitamins, and/or appropriate precursor may further be included. The culture medium and precursors may be added to the culture medium in a batch or continuous process, without being limited thereto.

In the present disclosure, during the culturing process of the microorganism, compounds such as ammonium hydroxide, potassium hydroxide, ammonia, phosphoric acid, and sulfuric acid may be properly added to the cultures in order to adjust the pH of the culture medium. Also, a defoaming agent such as fatty acid polyglycol ester may be added during culturing in order to inhibit formation of foams. In addition, oxygen or oxygen-containing gas may be injected into the culture medium to maintain the culture medium in an aerobic condition, and nitrogen, hydrogen, or carbon dioxide gases may be injected into the cultures to maintain the culture in anaerobic and micro-aerobic conditions without injecting any other gases therefor, but the embodiment is not limited thereto.

The temperature of the culture medium may be maintained at 25° C. to 50° C., more specifically at 30° C. to 37° C., without being limited thereto. The culturing may be continued until a desired amount of a product is obtained, specifically for 10 hours to 100 hours, without being limited thereto.

The target product produced by the culturing may be exported into the culture medium or remain in the cells.

The method may further include recovering the target product from the culture medium or the microorganism.

The recovering of the target product produced in the culturing step of the present disclosure may be performed by collecting the target product from the culture using any known method selected according to the culturing method. For example, centrifugation, filtration, anion-exchange chromatography, crystallization, and high-performance liquid chromatography (HPLC) may be used, and the target amino acid may be recovered from the culture medium or the microorganism using any appropriate method in the art, without being limited thereto. The culture may also be named culture broth.

Also, the recovering step may include a purification process which may be performed using an appropriate method well known in the art. Thus, the recovered target product may be a purified form or a fermentation broth of a microorganism including the target product (Introduction to Biotechnology and Genetic Engineering, A. J. Nair., 2008).

Another aspect of the present disclosure provides a method for producing methionine, the method including: producing O-acetylhomoserine by culturing a microorganism in a culture medium; and converting the O-acetylhomoserine into methionine by reacting the O-acetylhomoserine with a sulfide, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The SEQ ID NO: 1, the inner membrane protein YjeH, the variant thereof, the polynucleotide encoding the variant, the vector including the polynucleotide, the microorganism including the same, and O-acetylhomoserine are as described above.

In the present disclosure, O-acetylhomoserine, as a methionine precursor, may be converted into methionine via reaction with a sulfide.

The reaction with the sulfide refers to a process of generating L-methionine from O-succinyl homoserine using any known method. For example, L-methionine may be produced by reacting methyl mercaptan, as a sulfide, with O-acetylhomoserine. In addition, a catalyst or an enzyme may be added or reaction may be performed in a microorganism including an enzyme to increase reaction rates and increase yields.

The “sulfide” may be, for example, methyl mercaptan, and the methyl mercaptan may be any methyl mercaptan derivatives capable of providing sulfur atoms such as dimethylsulfide (DMS) disclosed in International Publication No. WO 2010/098629) as well as sodium methyl mercaptan (CH₃S—Na) in a liquid phase and methyl mercaptan (CH3SH) in a gaseous or liquid state.

As an enzyme used in methionine conversion reaction, cystathionine-γ-synthase, O-acetylhomoserine sulfhydrylase, or O-succinylhomoserine sulfhydrylase which are known in the art may be used.

A process of methionine conversion reaction of the present disclosure may be expressed as the following reaction scheme:

O-acetyl-L-homoserine+CH₃SH↔L-methionine+acetate

In the reaction, a CH₃S— residue of methyl mercaptan is substituted with an acetate residue of O-acetylhomoserine to produce L-methionine.

The reaction may be conducted at a temperature of 20° C. to 45° C. while maintaining the pH of a reaction medium in the range of 6 to 7, and pyridoxal 5′-phosphate may be added as a coenzyme.

The conversion of an L-methionine precursor into L-methionine in the presence of methyl mercaptan is an enzymatic reaction and is disclosed in International Publication No. WO 2008/013432. International Publication No. WO 2008/013432 provides details on properties and preparation of methionine-converting enzymes having an appropriate activity for converting an L-methionine precursor into L-methionine in the presence of methyl mercaptan. Such appropriate methionine-converting enzyme may be prepared using gene expression according to biotechnological methods.

A sequence of a gene encoding an enzyme having methionine converting activity may be obtained from, but not limited to, database of the NCBI (USA) and DNA data bank (KEGG, Japan). For biological conversion, the gene may be cloned from the obtained gene sequence, and then introduced into an expression vector. Specifically, the gene may be expressed in an active form from pCL-CJI vector (CJ, Korea) that is an expression vector for E. coli in recombinant strains, and an expressed protein may be obtained from an enzyme solution in which sonicated cells are lysed. Both the strain expressing the methionine-converting enzyme and the expressed methionine-converting enzyme may be directly used in the methionine conversion reaction.

The methionine-converting enzyme expressed from the gene or the microorganism strain expressing the same may be directly, partially, or entirely mixed with a fermentation supernatant or a fermentation broth in which an L-methionine precursor is accumulated to initiate the reaction. That is, the reaction may be initiated by adding the methionine-converting enzyme or the microorganism strain expressing the same to the fermentation solution in which O-acetylhomoserine is accumulated and adding methyl mercaptan thereto.

Specifically, O-acetylhomoserine that is accumulated in the fermentation solution may be converted into L-methionine by cystathionine-γ-synthase, O-acetylhomoserine sulfhydrylase, or O-succinyl homoserine sulfhydrylase.

The methionine conversion reaction may be confirmed using DTNB (5,5-dithiobis(2-nitro-benzoic acid), Sigma, USA), and the reaction product may be analyzed by HPLC. During the process of methionine conversion reaction, byproducts such as acetic acid may be additionally obtained, without a separate production process, via reaction between methyl mercaptan and O-acetylhomoserine.

The enzymatic reaction between L-methionine precursor and methyl mercaptan may be represented by the following reaction scheme:

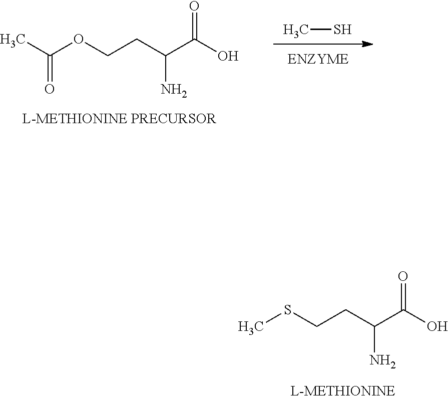

The method of producing methionine may be easily determined by one or ordinary skill in the art under known optimal culturing conditions and enzyme activation conditions. Specific culturing methods and media are as described above.

Another aspect of the present disclosure provides a method for producing glufosinate, the method including: producing O-acetylhomoserine or homoserine by culturing a microorganism in a culture medium; and converting the O-acetylhomoserine or homoserine into glufosinate, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The SEQ ID NO: 1, the inner membrane protein YjeH, the variant thereof, the polynucleotide encoding the variant, the vector including the polynucleotide, the microorganism including the same, and homoserine are as described above.

As used herein, the term “glufosinate” refers to a broad range of naturally occurring herbicides produced by several species of Streptomyces soil bacteria, and is also known as phosphinothricin.

In the present disclosure, O-acetylhomoserine or homoserine may be converted into glufosinate via a plurality of steps including halogenation.

The glufosinate conversion reaction is disclosed, for example, in CN 108516991 A. Specifically, the glufosinate conversion reaction may include: (1) a step of performing azeotropic dehydration of homoserine (Formula II) using an organic solvent in the presence of an acidic catalyst; and a step of adding a halogenating agent to the azeotropically dehydrated homoserine and performing halogenation to obtain L-3,6-bis(2-haloethyl)-2,5-dione piperazine (Formula III); (2) a step of adding methylphosphinic acid diester or methyl phosphite diester to the L-3,6-bis(2-haloethyl)-2,5-dione piperazine and performing an Abbov reaction using a high-boiling solvent in the presence of a catalyst to obtain a compound of Formula IV; and (3) a step of performing a hydrolysis reaction by dissolving the compound of Formula IV in an acid and heating the solution, removing a solvent after completion of the reaction, adding an alcohol thereto, and performing conversion reaction by adding an alkylene oxide thereto to obtain glufosinate (Formula I).

The glufosinate conversion reaction using homoserine as a precursor may be represented by the following reaction scheme:

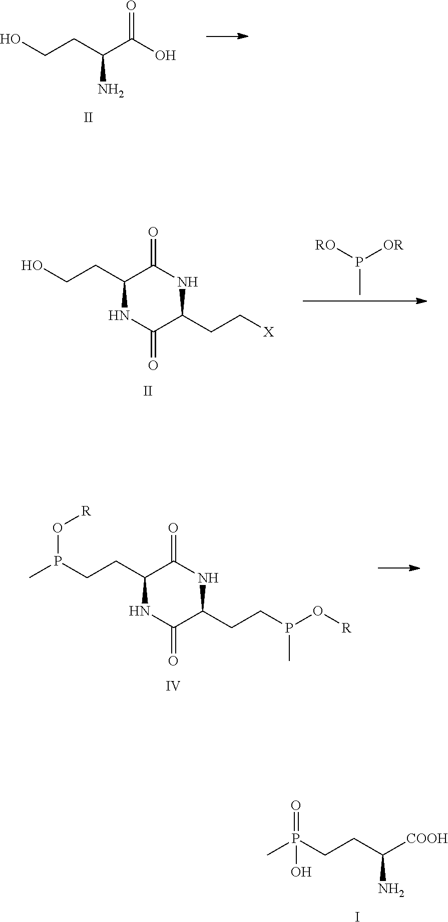

The method for producing glufosinate may be performed using homoserine prepared by biological fermentation as a precursor according to the present disclosure. Glufosinate may be produced by preparing L-3,6-bis(2-haloethyl)-2,5-dione piperazine by reaction with methylphosphonium by azeotropic dehydration and halogenation, performing an Abbov reaction, and then performing a hydrolysis reaction as described above.

Another aspect of the present disclosure provides a composition for producing homoserine including a microorganism or a culture of the microorganism, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

Another aspect of the present disclosure provides a composition for producing O-acetylhomoserine including a microorganism or culture of the microorganism, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The SEQ ID NO: 1, the inner membrane protein YjeH, the variant thereof, the polynucleotide encoding the variant, the vector including the polynucleotide, the microorganism including the same, homoserine, and O-acetylhomoserine are as described above.

The composition for producing O-acetylhomoserine or homoserine may refer to a composition used to produce O-acetylhomoserine or homoserine by the variant of an inner membrane protein YjeH of the present disclosure. The composition may include the variant of an inner membrane protein YjeH or a component capable of operating the variant of an inner membrane protein YjeH without limitation. The variant of an inner membrane protein YjeH may be in a form inserted into a vector capable of expressing an operably linked gene in a host cell.

The composition may further include a cryoprotectant or an excipient. The cryoprotectant or the excipient may be a naturally occurring substance or one which does not occur naturally, but is not limited thereto. As another example, the cryoprotectant or the excipient may be a substance that the microorganism does not naturally come into contact with or a substance that is not naturally included in the microorganism, but is not limited thereto.

Another aspect of the present disclosure provides a use of the variant of an inner membrane protein YjeH for producing O-acetylhomoserine or homoserine.

Another aspect of the present disclosure provides a use of a microorganism for producing O-acetylhomoserine or homoserine, wherein the microorganism includes at least any one of: a variant of an inner membrane protein YjeH including i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; a polynucleotide encoding the variant; and a vector including the polynucleotide.

The homoserine, O-acetylhomoserine, inner membrane protein YjeH, the variant thereof, the polynucleotide encoding the variant, the vector including the polynucleotide and the microorganism including the same are as described above.

### MODE FOR INVENTION

Hereinafter, the present disclosure will be described in more detail with reference to the following examples. However, the following examples are merely presented to exemplify the present disclosure, and the scope of the present disclosure is not limited thereto.

### Example 1: Construction of Plasmid

A plasmid (pDCM2, FIG. 1, SEQ ID NO: 37) was designed for insertion and replacement of a gene in the Corynebacterium chromosome and synthesized using a gene synthesis service of Bionics Co., Ltd. The plasmid was designed to contain a restriction enzyme efficient for cloning with reference to a paper related to the sacB system commonly known in the art (Gene, 145 (1994) 69-73). The synthesized pDCM2 plasmid has the following characteristics.
1) Due to a replication origin only active in E. coli, the plasmid self-replicates only in E. coli and cannot self-replicate in Corynebacterium.
2) The plasmid contains a kanamycin-resistance gene as a selection marker.
3) The plasmid contains Levan sucrose gene (sacB) as a secondary positive-selection marker.
4) No gene information derived from the pDCM2 plasmid remains in the ultimately prepared strain.

### Example 2: Construction of Strains Introduced with Variant Exogenous Membrane Protein (YjeH) and Evaluation of Ability to Produce O-Acetylhomoserine and Homoserine

2-1. Construction of Strains Introduced with Exogenous Membrane Protein (YjeH) and Variant YjeH

In order to evaluate effectiveness of YjeH that is introduced into Corynebacterium glutamicum ATCC13032 as an exogenous membrane protein and an O-acetylhomoserine-exporting protein, a vector for chromosomal insertion including yjeH gene (SEQ ID NO: 2) encoding YjeH derived from E. coli was prepared.

Specifically, in order to construct a transposase-deletion vector, a pair of primers (SEQ ID NOS: 4 and 5) for amplifying a 5′ upstream region and a pair of primers (SEQ ID NOS: 6 and 7) for amplifying a 3′ downstream region with respect to a gene encoding transposase (SEQ ID NO: 3, gene No. NCgl2335) were designed. The primers of SEQ ID NOS: 5 and 6 were designed to cross each other. A restriction enzyme Smal sequence was located in this region. The primer sequences are shown in Table 1 below.

TABLE 1

| SEQ ID | Sequence | |
|---|---|---|
| NO: | name | Sequence |
| SEQ ID | Tn_5 F | tgaattcgagctcggtacccCACCGACGC |
| NO: 4 | | GCATCTGCCT |
| SEQ ID | Tn_5 R | GGTGTGGTGACTTTCAGCAGTTCCCGGGG |
| NO: 5 | | GGGAGGAGGCATGTGGTGTTG |
| SEQ ID | Tn_3 F | CAACACCACATGCCTCCTCCCCCCCGGGA |
| NO: 6 | | ACTGCTGAAAGTCACCACACC |
| SEQ ID | Tn_3 R | gtcgactctagaggatccccCTCCCAAAC |
| NO: 7 | | CATTGAGGAATGG |

PCR was performed using the chromosome of wild-type (WT) ATCC13032 as a template and primers of SEQ ID NOS: 4 and 5 and SEQ ID NOS: 6 and 7. The PCR was performed under the following conditions. After denaturation at 95° C. for 5 minutes, 30 cycles of denaturation at 95° C. for 30 seconds, annealing at 55° C. for 30 seconds, and polymerization at 72° C. for 30 seconds were repeated, and then polymerization was performed at 72° C. for 7 minutes. As a result, DNA fragments of an 851 bp 5′ upstream region and an 847 bp 3′ downstream region with respect to the NCgl2335 gene deletion region were obtained.

PCR was performed using the two amplified DNA fragments as templates and primers of SEQ ID NOS: 4 and 7. PCR was performed under the following conditions. After denaturation at 95° C. for 5 minutes, 30 cycles of denaturation at 95° C. for 30 seconds, annealing at 55° C. for 30 seconds, and polymerization at 72° C. for 90 seconds were repeated, and then polymerization was performed at 72° C. for 7 minutes. As a result, a 1648 bp DNA fragment including a region capable of deleting the gene encoding transposase (SEQ ID NO: 3, gene No. NCgl2335) was amplified.

The obtained PCR products were fusion-cloned using a pDCM2 vector treated with a restriction enzyme Smal and an In-Fusion® HD Cloning Kit (Clontech). E. coli DH5α was transformed with the cloned vector, and the transformed E. coli was smeared on an LB solid medium containing kanamycin (25 mg/L). Colonies transformed with the plasmid inserted with the target gene were selected by PCR, and the plasmid was obtained using a plasmid extraction method. Finally, pDCM2-ΔNCgl2335 recombinant vector including the cloned NCgl2335 deletion cassette was constructed.

In order to evaluate effectiveness of an O-acetylhomoserine-exporting protein, a vector for chromosomal insertion including a gene (SEQ ID NO: 2) encoding YjeH derived from E. coli was prepared. To this end, a vector expressing yjeH gene was constructed using the CJ7 promoter (SEQ ID NO: 8, U.S. Pat. No. 7,662,943 B2). A pair of primers (SEQ ID NOS: 9 and 10) for amplifying the CJ7 promoter region and a pair of primers (SEQ ID NOS: 11 and 12) for amplifying the yjeH region of E. coli were designed. The primer sequences are shown in Table 2 below.

TABLE 2

| SEQ ID | Sequence | |
|---|---|---|
| NO: | name | Sequence |
| SEQ ID | CJ7_yjeH F | ACACCACATGCCTCCTCccc |
| NO: 9 | | AGAAACATCCCAGCGCTAC |
| SEQ ID | CJ7_yjeH R | AGTTCTTGTTTGAGTCCACT |
| NO: 10 | | CATAGTGTTTCCTTTCGTTG |
| | | GGT |
| SEQ ID | yjeH F | ACCCAACGAAAGGAAACACT |
| NO: 11 | | ATGAGTGGACTCAAACAAGA |
| | | ACTG |
| SEQ ID | yjeH R | GACTTTCAGCAGTTcccggg |
| NO: 12 | | TTATGTGGTTATGCCATTTT |
| | | CCGG |

PCR was performed using pECCG117-PCJ7-gfp (U.S. Pat. No. 7,662,943 B2) as a template and primers of SEQ ID NOS: 9 and 10 and PCR was performed using the chromosome of wild-type E. coli as a template and primers of SEQ ID NOS: 11 and 12, respectively. The PCR was performed under the following conditions. After denaturation at 95° C. for 5 minutes, 30 cycles of denaturation at 95° C. for 30 seconds, annealing at 55° C. for 30 seconds, and polymerization at 72° C. for 90 seconds were repeated, and then polymerization was performed at 72° C. for 7 minutes. As a result, a 360 bp DNA fragment of the CJ7 promoter region and a 1297 bp DNA fragment of the yjeH gene of E. coli were obtained.

PCR was performed using the two amplified DNA fragments as templates and primers of SEQ ID NOS: 9 and 12. The PCR was performed under the following conditions. After denaturation at 95° C. for 5 minutes, 30 cycles of denaturation at 95° C. for 30 seconds, annealing at 55° C. for 30 seconds, and polymerization at 72° C. for 90 seconds were repeated, and then polymerization was performed at 72° C. for 7 minutes. As a result, a 1614 bp DNA fragment including a region into which the CJ7 promoter and the yjeH gene were inserted was amplified.

The gene-deletion DNA fragment obtained by the PCR was cloned to pDCM2-ΔNCgl2335 vector treated with a restriction enzyme Smal using an In-Fusion® HD Cloning Kit (Clontech). Finally, pDCM2-ΔNCgl2335::PCJ7-yjeH (eco, WT) recombinant vector was constructed.

Specifically, phenylalanine (Phe), which is the 92^{nd} amino acid of the amino acid sequence of YjeH, was substituted with asparagine (Asn) (F92N) using the pDCM2-ΔNCgl2335::PCJ7-yjeH (eco, WT) plasmid as a template and primers of SEQ ID NOS: 13 and 14. A constructed plasmid including a gene encoding the variant YjeH (F92N) was named pDCM2-ΔNCgl2335::PCJ7-yjeH (eco, F92N).

Also, phenylalanine (Phe), which is the 351^{st} amino acid of the amino acid sequence of YjeH, was substituted with leucine (F351L) using the pDCM2-ΔNCgl2335::PCJ7-yjeH (eco, WT) plasmid as a template and primers of SEQ ID NOS: 15 and 16. A constructed plasmid including a gene encoding the variant YjeH (F351L) was named pDCM2-ΔNCgl2335::PCJ7-yjeH (eco, F351L). The primer sequences are shown in Table 3 below.

TABLE 3

| | Sequence | |
|---|---|---|
| SEQ ID NO: | name | Sequence |
| SEQ ID NO: 13 | F92N F | CGGCTGGCTGAATTTATCGGTCA |
| SEQ ID NO: 14 | F92N R | TGACCGATAAATTCAGCCAGCCG |
| SEQ ID NO: 15 | F351L F | CAATGGCATCCTTATTATGATTT |
| SEQ ID NO: 16 | F351L R | AAATCATAATAAGGATGCCATTG |

The ATCC13032 strain was transformed with each of the constructed pDCM2-ΔNCgl2335, pDCM2-ΔNCgl2335::PCJ7-yjeH (eco, WT), pDCM2-ΔNCgl2335::PCJ7-yjeH (eco, F92N), and pDCM2-ΔNCgl2335::PCJ7-yjeH (eco, F351L) using an electric-pulse method and subjected to a secondary crossover process to obtain ATCC13032 ΔNCgl2335, ATCC13032 ΔNCgl2335::PCJ7-yjeH (eco, WT), ATCC13032 ΔNCgl2335::PCJ7-yjeH (eco, F92N), and ATCC13032 ΔNCgl2335::PCJ7-yjeH (eco, F351L) in which the NCgl2335 gene was deleted from the chromosome. Insertion of inactivated NCgl2335 gene and newly introduced E. coli yjeH gene was identified by performing PCR using the primers of SEQ ID NOS: 4 and 7 and comparing results with those of ATCC13032 in which the NCgl2335 gene was not inactivated.

2-2. Evaluation of Ability to Produce O-Acetylhomoserine

In order to compare the O-acetylhomoserine (O-AH) producing ability of ATCC13032 ΔNCgl2335, ATCC13032 ΔNCgl2335::PCJ7-yjeH (eco, WT), ATCC13032 ΔNCgl2335::PCJ7-yjeH (eco, F92N), and ATCC13032 ΔNCgl2335::PCJ7-yjeH (eco, F351L), which were constructed in Example 2-1, with that of the wild-type strain ATCC13032, O-acetylhomoserine contained in the cultures was analyzed according to the following method.

One inoculation loop of each of the strains was inoculated to a 250 mL corner-baffled flask containing 25 mL of a medium below and then cultured while shaking at 33° C. for 20 hours at 200 rpm. The concentrations of O-acetylhomoserine were analyzed using HPLC, and the analyzed concentrations are shown in Table 4.

O-Acetylhomoserine Production Medium (pH 7.2)

30 g of glucose, 2 g of KH₂PO₄, 3 g of urea, 40 g of (NH₄)₂SO₄, 2.5 g of peptone, 5 g (10 mL) of corn steep liquor (CSL, Sigma), 0.5 g of MgSO₄·7H₂O, and 20 g of CaCO₃ (based on 1 L of distilled water)

TABLE 4

| Strain name | O-Acetylhomoserine (g/L) |
|---|---|
| ATCC13032 | 0.3 |
| ATCC13032 ΔNCgI2335 | 0.3 |
| ATCC13032 ΔNCgI2335::PCJ7-yjeH | 0.7 |
| (eco, WT) | |
| ATCC13032 ΔNCgI2335::PCJ7-yjeH | 1.2 |
| (eco, F92N) | |
| ATCC13032 ΔNCgI2335::PCJ7-yjeH | 1.0 |
| (eco, F351L) | |

As a result, it was confirmed that O-acetyl-L-homoserine was accumulated with a concentration of 0.3 g/L when the ATCC13032, as a control, was cultured, and production of O-acetyl-L-homoserine was not affected by deletion of the NCgl2335 gene (transposase), as shown in Table 4. Particularly, it was confirmed that O-acetyl-L-homoserine was accumulated with a concentration of 0.7 g/L when the wild-type yjeH gene was expressed, and O-acetyl-L-homoserine was accumulated with concentrations of 1.2 g/L and 1.0 g/L when the variant yjeH genes were expressed.

Therefore, based on the results shown above, it was confirmed that the wild-type and variant yjeH genes of the present disclosure significantly increased production of the amino acid of interest by exporting O-acetylhomoserine from the ATCC13032.

The ATCC13032 ΔNCgl2335::PCJ7-yjeH (eco, F92N) and ATCC13032 ΔNCgl2335::PCJ7-yjeH (eco, F351L) strains were named Corynebacterium glutamicum CM04-0651 and Corynebacterium glutamicum CM04-0652, respectively, and deposited at the Korean Culture Center of Microorganisms under the Budapest Treaty on Dec. 4, 2019, with Accession Nos. KCCM12633P and KCCM12634P, respectively.

2-3. Evaluation of Ability to Produce Homoserine

In order to compare the homoserine producing ability of ATCC13032 ΔNCgl2335, ATCC13032 ΔNCgl2335::PCJ7-yjeH (eco, WT), ATCC13032 ΔNCgl2335::PCJ7-yjeH (eco, F92N), and ATCC13032 ΔNCgl2335::PCJ7-yjeH (eco, F351L), which were constructed in Example 2-1, with that of the wild-type strain ATCC13032, homoserine contained in the cultures was analyzed according to the following method.

One inoculation loop of each of the strains was inoculated to a 250 mL corner-baffled flask containing 25 mL of a medium below and then cultured while shaking at 33° C. for 20 hours at 200 rpm. The concentrations of homoserine were analyzed using HPLC, and the analyzed concentrations are shown in Table 5.

Homoserine Production Medium (pH 7.2)

30 g of glucose, 2 g of KH₂PO₄, 3 g of urea, 40 g of (NH₄)₂SO₄, 2.5 g of peptone, 5 g (10 mL) of corn steep liquor (CSL, Sigma), 0.5 g of MgSO₄·7H₂O, and 20 g of CaCO₃ (based on 1 L of distilled water)

TABLE 5

| Strain name | Homoserine (g/L) |
|---|---|
| ATCC13032 | 0.0 |
| ATCC13032 ΔNCgI2335 | 0.0 |
| ATCC13032 ΔNCgI2335::PCJ7-yjeH | 0.0 |
| (eco, WT) | |
| ATCC13032 ΔNCgI2335::PCJ7-yjeH | 0.2 |
| (eco, F92N) | |
| ATCC13032 ΔNCgI2335::PCJ7-yjeH | 0.2 |
| (eco, F351L) | |

As a result, it was confirmed that homoserine was not accumulated with a concentration of 0.0 g/L when the ATCC13032, as a control, was cultured, and production of homoserine was not affected by deletion of the NCgl2335 gene (transposase) as shown in Table 5. Particularly, it was also confirmed that homoserine was not accumulated with a concentration of 0.0 g/L when the wild-type yjeH gene was expressed, and homoserine was accumulated with concentrations of 0.2 g/L and 0.2 g/L when the variant yjeH genes were expressed.

Therefore, based on the results shown above, it was confirmed that the variant yjeH gene of the present disclosure increased production of the amino acid of interest by exporting homoserine from the ATCC13032.

### Example 3: Construction of Strain with Enhanced Ability to Produce O-Acetylhomoserine and Evaluation of Ability to Produce O-Acetylhomoserine and Homoserine

3-1. Inactivation of Cystathionine Gamma (γ)-synthase (MetB)

Via PCR using chromosomal DNA of Corynebacterium glutamicum ATCC13032 as a template, metB gene encoding cystathionine γ-synthase involved in a degradation pathway of O-acetylhomoserine was obtained. Information on a nucleotide sequence of the metB gene (NCBI No. Ncgl2360, SEQ ID NO: 17) was obtained from GenBank of the National Institutes of Health (USA), and primers (SEQ ID NOS: 18 and 19) including the N-terminus of the metB gene and a linker and primers (SEQ ID NOS: 20 and 21) including the C-terminus and the linker were synthesized based thereon. The primer sequences are shown in Table 6 below.

TABLE 6

| SEQ ID | Sequence | |
|---|---|---|
| NO | name | Sequence |
| SEQ ID | metB_N_del F | TCTAGACGCCCGCATACTG |
| NO: 18 | | GCTTC |
| SEQ ID | metB_N_del R | CCCATCCACTAAACTTAAA |
| NO: 19 | | CAGATGTGATCGCCCGGC |
| SEQ ID | metB_C_del F | TGTTTAAGTTTAGTGGATG |
| NO: 20 | | GGGAAGAACCACCCAGGCC |
| SEQ ID | metB_C_del R | GTCGACCAATCGTCCAGAG |
| NO: 21 | | GGCG |

PCR was performed using the chromosomal DNA of ATCC13032 as a template and primers of SEQ ID NOS: 18 and 19 and SEQ ID NOS: 20 and 21. PfuUltra™ high-reliability DNA polymerase (Stratagene) was used as a polymerase, and PCR was performed by repeating 30 cycles of denaturation at 96° C. for 30 seconds, annealing at 53° C. for 30 seconds, and polymerization at 72° C. for 1 minute. As a result, an amplified 558 bp gene containing the N-terminus of the metB gene and the linker and an amplified 527 bp gene containing the C-terminus of the metB gene and the linker were obtained, respectively.

PCR was performed using the two amplified genes obtained as described above as templates by repeating 10 cycles of denaturation at 96° C. for 60 seconds, annealing at 50° C. for 60 seconds, and polymerization at 72° C. for 1 minute, and then adding the primers of SEQ ID NOS: 18 and 21 thereto, followed by polymerization 20 times. As a result, a 1064 bp inactivation cassette including the N-terminus-linker-C-terminus of the metB gene was obtained. The obtained PCR products were treated with restriction enzymes XbaI and SalI and ligated to the pDCM2 vector treated with the restriction enzymes XbaI and SalI for cloning to ultimately construct a pDCM2-ΔmetB recombinant vector cloned with the metB inactivation cassette.

The ATCC13032 was transformed with the constructed pDCM2-ΔmetB vector using an electric-pulse method and subjected to a secondary crossover process to obtain ATCC13032 ΔmetB in which the metB gene was inactivated in the chromosome. The inactivated metB gene was ultimately confirmed by comparison with ATCC13032 in which the metB gene was not inactivated after performing PCR using the primers of SEQ ID NOS: 18 and 21.

3-2. Inactivation of O-Acetylhomoserine (thiol)-lyase (MetY)

Via PCR using chromosomal DNA of Corynebacterium glutamicum ATCC13032 as a template, metY gene encoding O-acetylhomoserine (thiol)-lyase involved in a degradation pathway of O-acetylhomoserine was obtained. Information on a nucleotide sequence of the metY gene (NCBI No. Ncg10625, SEQ ID NO: 22) was obtained from GenBank of the National Institutes of Health (USA), and primers (SEQ ID NOS: 23 and 24) including the N-terminus of the metY gene and a linker and primers (SEQ ID NOS: 25 and 26) including the C-terminus and a linker were synthesized based thereon. The primer sequences are shown in Table 7 below.

TABLE 7

| SEQ ID | Sequence | |
|---|---|---|
| NO | name | Sequence |
| SEQ ID | metY_N_del F | TCTAGACCATCCTGCACCA |
| NO: 23 | | TTTAG |
| SEQ ID | metY_N_del R | CCCATCCACTAAACTTAAA |
| NO: 24 | | CACGCTCCTGCCAGGTTC |
| SEQ ID | metY_C_del F | TGTTTAAGTTTAGTGGATG |
| NO: 25 | | GGCTTGGTACGCAACCAAG |
| | | G |
| SEQ ID | metY_C_del R | GTCGACGATTGCTCCGGCT |
| NO: 26 | | TCGG |

PCR was performed using the chromosomal DNA of ATCC13032 as a template and primers of SEQ ID NOS: 23 and 24 and SEQ ID NOS: 25 and 26. PfuUltra™ high-reliability DNA polymerase (Stratagene) was used as a polymerase, and PCR was performed by repeating 30 cycles of denaturation at 96° C. for 30 seconds, annealing at 53° C. for 30 seconds, and polymerization at 72° C. for 1 minute. As a result, an amplified 548 bp gene containing the N-terminus of the metY gene and the linker and an amplified 550 bp gene containing the C-terminus of the metY gene and the linker were obtained, respectively. PCR was performed using the two amplified genes obtained as described above as templates by repeating 10 cycles of denaturation at 96° C. for 60 seconds, annealing at 50° C. for 60 seconds, and polymerization at 72° C. for 1 minute, and then adding the primers of SEQ ID NOS: 23 and 26 thereto, followed by polymerization 20 times. As a result, a 1077 bp inactivation cassette including the N-terminus-linker-C-terminus of the metY gene was obtained. The obtained PCR products were treated with restriction enzymes XbaI and SalI and ligated to the pDCM2 vector treated with the restriction enzymes XbaI and SalI for cloning to ultimately construct a pDCM2-ΔmetY recombinant vector cloned with the metY inactivation cassette.

The ATCC13032 ΔmetB strain was transformed with the constructed pDCM2-ΔmetY vector using an electric-pulse method and subjected to a secondary crossover process to obtain ATCC13032 ΔmetB ΔmetY in which the metY gene is inactivated in the chromosome. The inactivated metY gene was ultimately confirmed by comparison with ATCC13032 in which the metY gene was not inactivated after performing PCR using the primers of SEQ ID NOS: 23 and 26.

3-3. Introduction of Variant Aspartokinase (LysC)

In order to maximize production of O-acetylhomoserine, Corynebacterium glutamicum ATCC13032-derived IysC gene (SEQ ID NO: 27) encoding aspartokinase was introduced with mutation (L377K) for enhancement of expression of the IysC gene and relief of feedback inhibition by L-lysine and L-threonine (U.S. Ser. No. 10/662,450 B2). In order to prepare a vector including the variant IysC gene, a pair of primers (SEQ ID NOS: 28 and 29) for amplifying a 5′ upstream region and a pair of primers (SEQ ID NOS: 30 and 31) for amplifying a 3′ downstream region with respect to the mutation position were designed. XbaI and SalI restriction enzyme regions were inserted to both ends of the primers of SEQ ID NOS: 28 and 31, and the primers of SEQ ID NOS: 29 and 30 were designed to cross each other, and nucleotide substitution was located at this region. Sequences of the primers are as shown in Table 8 below.

TABLE 8

| SEQ ID | | |
|---|---|---|
| NO | Sequence name | Sequence |
| SEQ ID | lysC_L377K_5 F | TCCTCTAGAGCTGCG |
| NO: 28 | | CAGTGTTGAATACG |
| SEQ ID | lysC_L377K_5 R | AGGTGGAAATCTTTT |
| NO: 29 | | CGATGTTC |
| SEQ ID | lysC_L377K_3 F | GAACATCGAAAAGAT |
| NO: 30 | | TTCCACCT |
| SEQ ID | lysC_L377K_3 R | GACTCTAGAGTTCAC |
| NO: 31 | | CTCAGAGACGATTA |

PCR was performed using the chromosome of Corynebacterium glutamicum ATCC13032 as a template and primers of SEQ ID NOS: 28 and 29 and SEQ ID NOS: 30 and 31. PCR was performed under the following conditions. After denaturation at 95° C. for 5 minutes, 30 cycles of denaturation at 95° C. for 30 seconds, annealing at 55° C. for 30 seconds, and polymerization at 72° C. for 30 seconds were repeated, and then polymerization was performed at 72° C. for 7 minutes. As a result, a 512 bp DNA fragment of a 5′ upstream region and a 522 bp DNA fragment of a 3′ downstream region with respect to the mutation of the IysC gene were obtained.

PCR was performed using the two amplified DNA fragments as templates and primers of SEQ ID NOS: 28 and 31. PCR was performed under the following conditions. After denaturation at 95° C. for 5 minutes, 30 cycles of denaturation at 95° C. for 30 seconds, annealing at 55° C. for 30 seconds, and polymerization at 72° C. for 60 seconds were repeated, and then polymerization was performed at 72° C. for 7 minutes. As a result, a 1011 bp DNA fragment including the variant IysC (L377K) gene encoding an aspartokinase variant in which leucine at the 377th position was substituted with lysine was amplified.

The IysC (L377K) fragment obtained by the PCR was treated with restriction enzymes XbaI and SalI at both ends and ligated to the pDCM2 vector treated with the restriction enzymes XbaI and SalI, followed by cloning to ultimately obtain a pDCM2-lysC (L377K) recombinant vector cloned with IysC (L377K) substitution cassette.

The ATCC13032 ΔmetB ΔmetY strain was transformed with the prepared pDCM2-lysC (L377K) vector by an electric-pulse method and subjected to a second crossover process to obtain Corynebacterium glutamicum ATCC 13032 ΔmetB ΔmetY lysC (L377K) introduced with the variant IysC gene on the chromosome. The variant gene was ultimately identified by PCR performed using the primers of SEQ ID NOS: 28 and 31 and then comparing the sequence with that of the wild-type IysC gene by sequencing.

3-4. Construction of Plasmid Introduced with O-Acetylhomoserine Transferase (MetX)

In order to amplify a gene encoding O-acetylhomoserine transferase (MetX), information on a nucleotide sequence of metX gene (NCBI No. NCgl0624, SEQ ID NO: 32) was obtained from GenBank of the National Institutes of Health (USA), a BamHI restriction enzyme site was inserted into both ends of each of primers (SEQ ID NOS: 33 and 34) for amplification from a promoter region (located about 300 bp upstream from a start codon) to a terminator region (located about 100 bp downstream from a stop codon) based thereon. PCR was performed under the following conditions. After denaturation at 95° C. for 5 minutes, 30 cycles of denaturation at 95° C. for 30 seconds, annealing at 55° C. for 30 seconds, and polymerization at 72° C. for 90 seconds were repeated, and then polymerization was performed at 72° C. for 7 minutes. As a result, a 1546 bp DNA fragment of a coding region of the metX gene was obtained. A pECCG117 vector (KR 10-0057684 B1) and the metX DNA fragment were treated with the restriction enzyme BamHI, ligated using a DNA ligase, and cloned to obtain a plasmid which was named pECCG117-metX WT. Sequences of the primers are as shown in Table 9 below.

TABLE 9

| SEQ ID | Sequence | |
|---|---|---|
| NO | name | Sequence |
| SEQ ID | metX F | GGATCCCCTCGTTGTTCA |
| NO: 33 | | CCCAGCAACC |
| SEQ ID | metX R | GGATCCCAAAGTCACAAC |
| NO: 34 | | TACTTATGTTAG |

3-5. Construction of O-Acetylhomoserine-producing Strain and Evaluation of Ability to Produce O-Acetylhomoserine

After the pECCG117-metX WT vector prepared in Example 3-4 was introduced into Corynebacterium glutamicum ATCC13032 ΔmetB ΔmetY lysC (L377K) using an electric-pulse method, the strain was smeared on a selective medium containing kanamycin (25 mg/L) and cultured to obtain transformants.

For comparison of the O-acetylhomoserine producing ability of the strains, the strains were cultured by way of the following method, and O-acetylhomoserine contained in the cultures was analyzed.

One inoculation loop of each of the strains was inoculated to a 250 mL corner-baffled flask containing 25 mL of a medium below and then cultured while shaking at 37° C. for 20 hours at 200 rpm. The concentrations of O-acetylhomoserine were analyzed using HPLC, and the analyzed concentrations are shown in Table 10.

O-Acetylhomoserine Production Medium (pH 7.2)

30 g of glucose, 2 g of KH₂PO₄, 3 g of urea, 40 g of (NH₄)₂SO₄, 2.5 g of peptone, 5 g (10 mL) of corn steep liquor (CSL, Sigma), 0.5 g of MgSO₄·7H₂O, 400 mg of methionine, and 20 g of CaCO₃ (based on 1 L of distilled water)

TABLE 10

| Strains | O-AH (g/L) |
|---|---|
| ATCC13032 ΔmetB ΔmetY lysC (L377K) | 0.7 |
| ATCC13032 ΔmetB ΔmetY lysC (L377K)/ | 1.3 |
| pECCG117-metX WT | |

As a result, it was confirmed that O-acetyl-L-homoserine was accumulated with a concentration of 0.7 g/L when the ATCC13032 ΔmetB ΔmetY lysC (L377K), as a control, was cultured and O-acetyl-L-homoserine was accumulated with a concentration 1.3 g/L when the ATCC13032 ΔmetB ΔmetY lysC (L377K)/pECCG117-metX WT strain was cultured as shown in Table 10.

Therefore, an increase in production of O-acetyl-L-homoserine was confirmed in the ATCC13032 ΔmetB ΔmetY lysC (L377K)/pECCG117-metX WT strain based on the results shown above.

3-6. Evaluation of Ability to Produce Homoserine

Corynebacterium glutamicum ATCC13032 ΔmetB ΔmetY lysC (L377K) constructed in Example 3-3 was introduced with the pECCG117-metX WT vector constructed in Example 3-4 using an electric-pulse method and smeared on a selective medium containing kanamycin (25 mg/L) and cultured to obtain transformants.

For comparison of the homoserine producing ability of the strains, the strains were cultured by way of the following method, and homoserine contained in the cultures was analyzed.

One inoculation loop of each of the strains was inoculated to a 250 mL corner-baffled flask containing 25 mL of a medium below and then cultured while shaking at 37° C. for 20 hours at 200 rpm. The concentrations of homoserine were analyzed using HPLC, and the analyzed concentrations are shown in Table 11.

Homoserine Production Medium (pH 7.2)

30 g of glucose, 2 g of KH₂PO₄, 3 g of urea, 40 g of (NH₄)₂SO₄, 2.5 g of peptone, 5 g (10 mL) of corn steep liquor (CSL, Sigma), 0.5 g of MgSO₄·7H₂O, 400 mg of methionine, and 20 g of CaCO₃ (based on 1 L of distilled water)

TABLE 11

| | Homoserine |
|---|---|
| Strains | production (g/L) |
| ATCC13032 ΔmetB ΔmetY lysC (L377K) | 0.2 |
| ATCC13032 ΔmetB ΔmetY lysC (L377K)/ | 0.2 |
| pECCG117-metX WT | |

As a result, it was confirmed that homoserine was accumulated with a concentration of 0.2 g/L when the ATCC13032 ΔmetB ΔmetY lysC (L377K), as a control, was cultured and homoserine was accumulated with a concentration of 0.2 g/L when the ATCC13032 ΔmetB ΔmetY lysC (L377K)/pECCG117-metX WT strain was cultured as shown in Table 11.

Therefore, based on the results, it was confirmed the ATCC13032 ΔmetB ΔmetY lysC (L377K)/pECCG117-metX WT strain had the same level of homoserine production.

### Example 4: Construction of Strain Introduced with Variant Exogenous Membrane Protein (YjeH) Having Enhanced Ability to Produce O-Acetylhomoserine and Evaluation of Ability to Produce O-Acetylhomoserine

4-1. Introduction of Variant Exogenous Membrane Protein (YjeH)

Corynebacterium glutamicum ATCC13032 ΔmetB ΔmetY lysC (L377K) strain constructed in Example 3-3 was transformed with the pDCM2-ΔNCgl2335, pDCM2-ΔNCgl2335::PCJ7-yjeH (eco, WT), pDCM2-ΔNCgl2335::PCJ7-yjeH (eco, F92N), and pDCM2-ΔNCgl2335::PCJ7-yjeH (eco, F351 L) vectors previously constructed in Example 2-1 and subjected to a secondary crossover process to obtain ATCC13032 ΔmetB ΔmetY lysC (L377K) ΔNCgl2335, ATCC13032 ΔmetB ΔmetY lysC (L377K) ΔNCgl2335::PCJ7-yjeH (eco, WT), ATCC13032 ΔmetB ΔmetY lysC (L377K) ΔNCgl2335::PCJ7-yjeH (eco, F92N), and ATCC13032 ΔmetB ΔmetY lysC (L377K) ΔNCgl2335::PCJ7-yjeH (eco, F351L) from which the NCgl2335 gene (transposase) was deleted from the chromosome. Insertion of inactivated NCgl2335 gene and newly introduced E. coli yjeH gene was identified by performing PCR using the primers of SEQ ID NOS: 4 and 7 and comparing results with those of ATCC13032 in which the NCgl2335 gene was not inactivated.

4-2. Construction of O-Acetylhomoserine-producing Strain and Evaluation of Ability to Produce O-Acetylhomoserine

After the pECCG117-metX WT vector prepared in Example 3-4 was introduced into ATCC13032 ΔmetB ΔmetY lysC (L377K) ΔNCgl2335, ATCC13032 ΔmetB ΔmetY lysC (L377K) ΔNCgl2335::PCJ7-yjeH (eco, WT), ATCC13032 ΔmetB ΔmetY lysC (L377K) ΔNCgl2335::PCJ7-yjeH (eco, F92N), and ATCC13032 ΔmetB ΔmetY lysC (L377K) ΔNCgl2335::PCJ7-yjeH (eco, F351L) prepared in Example 4-1 using an electric-pulse method, the strains were smeared on a selective medium containing kanamycin (25 mg/L) and cultured to obtain transformants.

For comparison of the O-acetylhomoserine producing ability of the strains, the strains were cultured by way of the following method, and O-acetylhomoserine contained in the cultures was analyzed.

One inoculation loop of each of the strains was inoculated to a 250 mL corner-baffled flask containing 25 mL of the O-acetylhomoserine production medium (pH 7.2) of Example 3-5 and then cultured while shaking at 33° C. for 20 hours at 200 rpm. The concentrations of O-acetylhomoserine were analyzed using HPLC, and the analyzed concentrations are shown in Table 12.

TABLE 12

| Strains | | O-AH (g/L) |
|---|---|---|
| ATCC13032 ΔmetB ΔmetY | — | 1.3 |
| lysC (L377K)/pECCG117- | ΔNCgI2335 | 1.3 |
| metX WT | ΔNCgI2335::PCJ7-yjeH | 2.1 |
| | (eco, WT) | |
| | ΔNCgI2335::PCJ7-yjeH | 2.7 |
| | (eco, F92N) | |
| | ΔNCgI2335::PCJ7-yjeH | 2.3 |
| | (eco, F351L) | |

As a result, it was confirmed that O-acetyl-L-homoserine was accumulated with a concentration of 1.3 g/L when Corynebacterium glutamicum ATCC13032 ΔmetB ΔmetY lysC (L377K), as a control, was cultured and production of O-acetyl-L-homoserine was not affected by deletion of the NCgl2335 gene (transposase) as shown in Table 12. Particularly, it was confirmed that O-acetyl-L-homoserine was accumulated with a concentration of 2.1 g/L when the wild-type yjeH gene was expressed and O-acetyl-L-homoserine was accumulated with concentrations of 2.7 g/L and 2.3 g/L when the variant yjeH genes were expressed.

Therefore, based on the results shown above, it was confirmed that the wild-type and variant yjeH genes of the present disclosure significantly increased production of the amino acid of interest by exporting O-acetyl-L-homoserine from the Corynebacterium glutamicum ATCC13032.

4-3. Evaluation of Ability to Produce Homoserine

After the pECCG117-metX WT vector prepared in Example 3-4 was introduced into ATCC13032 ΔmetB ΔmetY lysC (L377K) ΔNCgl2335, ATCC13032 ΔmetB ΔmetY lysC (L377K) ΔNCgl2335::PCJ7-yjeH (eco, WT), ATCC13032 ΔmetB ΔmetY lysC (L377K) ΔNCgl2335::PCJ7-yjeH (eco, F92N), and ATCC13032 ΔmetB ΔmetY lysC (L377K) ΔNCgl2335::PCJ7-yjeH (eco, F351L) prepared in Example 4-1 using an electric-pulse method, the strains were smeared on a selective medium containing kanamycin (25 mg/L) and cultured to obtain transformants.

For comparison of the homoserine producing ability of the strains, the strains were cultured by way of the following method, and homoserine contained in the cultures was analyzed.

One inoculation loop of each of the strains was inoculated to a 250 mL corner-baffled flask containing 25 mL of a medium below and then cultured while shaking at 33° C. for 20 hours at 200 rpm. The concentrations of homoserine were analyzed using HPLC, and the analyzed concentrations are shown in Table 13.

Homoserine Production Medium (pH 7.2)

30 g of glucose, 2 g of KH₂PO₄, 3 g of urea, 40 g of (NH₄)₂SO₄, 2.5 g of peptone, 5 g (10 mL) of corn steep liquor (CSL, Sigma), 0.5 g of MgSO₄·7H₂O, 400 mg of methionine, and 20 g of CaCO₃ (based on 1 L of distilled water)

TABLE 13

| Strains | | Homoserine (g/L) |
|---|---|---|
| ATCC13032 ΔmetB ΔmetY | — | 0.2 |
| lysC (L377K)/pECCG117- | ΔNCgI2335 | 0.2 |
| metX WT | ΔNCgI2335::PCJ7-yjeH | 0.2 |
| | (eco, WT) | |
| | ΔNCgI2335::PCJ7-yjeH | 1.2 |
| | (eco, F92N) | |
| | ΔNCgI2335::PCJ7-yjeH | 0.7 |
| | (eco, F351L) | |

As a result, it was confirmed that homoserine was accumulated with a concentration of 0.2 g/L when Corynebacterium glutamicum ATCC13032 ΔmetB ΔmetY lysC (L377K), as a control, was cultured, and production of homoserine was not affected by deletion of the NCgl2335 gene (transposase) as shown in Table 13. Particularly, it was confirmed that homoserine was accumulated with a concentration of 0.2 g/L when the wild-type yjeH gene was expressed, and homoserine was accumulated with concentrations of 1.2 g/L and 0.7 g/L when the variant yjeH genes were expressed.

Therefore, based on the results shown above, it was confirmed that the wild-type and variant yjeH genes of the present disclosure significantly increased production of the amino acid of interest by exporting homoserine from the Corynebacterium glutamicum ATCC13032.

### Example 5: Production of Methionine (MET) Using Conversion Reaction of O-Acetylhomoserine Produced by Strain

After the pECCG117-metX WT vector prepared in Example 3-4 was introduced into ATCC13032 ΔmetB ΔmetY lysC (L377K) ΔNCgl2335, ATCC13032 ΔmetB ΔmetY lysC (L377K) ΔNCgl2335::PCJ7-yjeH (eco, WT), ATCC13032 ΔmetB ΔmetY lysC (L377K) ΔNCgl2335::PCJ7-yjeH (eco, F92N), and ATCC13032 ΔmetB ΔmetY lysC (L377K) ΔNCgl2335::PCJ7-yjeH (eco, F351L) prepared in Example 4-1 using an electric-pulse method, the strains were smeared on a selective medium containing kanamycin (25 mg/L) and cultured to obtain transformants.

Also, according to a method disclosed in a previously filed patent document (WO2008/013432), metZ gene encoding O-succinylhomoserine sulfhydrylase derived from Pseudomonas aeruginosa and Pseudomonas putida, which would be used for conversion of O-acetylhomoserine into methionine, was cloned. PCR was performed by using the chromosome of each strain as a template and primers to obtain the metZ gene of Pseudomonas aeruginosa and Pseudomonas putida as follows: 30 cycles of denaturation at 94° C. for 30 seconds, annealing at 55° C. for 30 seconds, extension at 72° C. for 2 minutes to obtain DNA fragments. The obtained DNA fragments were digested with NdeI/PacI and cloned into pCL-CJ1 vector (CJ, Korea) digested with the same enzymes. E. coli W3110 cells were transformed with the cloned vector and cultured in an LB plate medium containing 50 μg/L spectinomycin, and then colonies were selected. The selected colonies were inoculated onto 3 mL of an LB medium containing 50 μg/L spectinomycin and cultured overnight at 37° C. The cultured cells were recovered, washed with a 0.1 M potassium phosphate buffer (pH 7.5), suspended in 200 μL of the potassium phosphate buffer, and lysed by sonication 5 times at 30 seconds intervals. The cell lysate was centrifuged at 12,000 rpm for 10 minutes and the supernatant was obtained to quantify the total protein level by using a Bio-Rad protein quantification solution (Bio-Rad, USA). Protein expression was identified by SDS-PAGE. The supernatant obtained from the cell extract was used for enzymatic conversion reaction.

Also, metZ gene encoding O-succinylhomoserine sulfhydrylase derived from Hyphomonas neptunium was cloned. PCR was performed using the chromosome of Hyphomonas neptunium as a template and a primer in the same manner as described above to obtain DNA fragments. The obtained DNA fragments were digested with NdeI/AvrII and cloned into pCL-CJ1 vector (CJ, Korea) digested with the same enzymes. A supernatant was obtained using the cloned vector was used to in the same manner as described above and used for enzymatic conversion reaction.

Each of the W3110 strains transformed with the metZ expression vector derived from Pseudomonas and transformed with the metZ expression vector derived from Hyphomonas were inoculated onto an LB plate medium containing spectinomycin, as an antibiotic, and cultured overnight at a temperature of 30° C. to 40° C. Then, a single colony was inoculated onto 40 mL of the LB medium containing spectinomycin and cultured for 5 hours at a temperature of 30° C. to 40° C. The cultured Pseudomonas-derived metZ-expressing strain and Hypomononas-derived metZ-expressing were cultured in a 1 L fermentation bath at 600 rpm to 900 rpm, at a temperature of 30° C. to 40° C., for 15 to 30 hours to obtain fermentation broths. The culture medium used in the culturing is as follows. The cells of the obtained fermentation broth were lysed by sonication to prepare enzyme solutions.

2XYT Medium

10 g of yeast extract, 16 g of tryptophan, 40 g of glucose, and 50 g of spectinomycin (based on 1 L of distilled water)

Methionine conversion reaction was performed by adding the Pseudomonas-derived and Hyphomonas-derived O-succinylhomoserine sulfhydrylase enzyme solution to the culture of the transformant producing O-acetylhomoserine. After adding 0.1 L of the enzyme solution was added to 2.0 L of the culture of O-acetylhomoserine from which cells were not removed, and 0.3 L of a 15% sodium methyl mercaptan solution was added thereto to initiate reaction. After 2 hours, the fermentation broth was recovered, cells were removed therefrom, and concentrations of the produced methionine were identified by HPLC. The analyzed concentrations are as shown in Table 14.

TABLE 14

| Strains | | Met (g/L) |
|---|---|---|
| ATCC13032 ΔmetB ΔmetY | — | 1.2 |
| lysC (L377K)/pECCG117- | ΔNCgI2335 | 1.2 |
| metX WT | ΔNCgI2335::PCJ7-yjeH | 1.9 |
| | (eco, WT) | |
| | ΔNCgI2335::PCJ7-yjeH | 2.5 |
| | (eco, F92N) | |
| | ΔNCgI2335::PCJ7-yjeH | 2.1 |
| | (eco, F351L) | |

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention. Therefore, the scope of the disclosure is defined not by the detailed description, but by the claims and their equivalents, and all variations within the scope of the claims and their equivalents are to be construed as being included in the disclosure.

1. A variant of an inner membrane protein YjeH comprising i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1.

2. The variant according to claim 1, wherein the variant has an ability to export O-acetylhomoserine or homoserine.

3. A polynucleotide encoding the variant of claim 1.

4. A vector comprising the polynucleotide of claim 3.

5. A microorganism comprising at least any one of: a variant of an inner membrane protein YjeH comprising i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; and a polynucleotide encoding the variant.

6. The microorganism according to claim 5, wherein the microorganism has an enhanced ability to produce O-acetylhomoserine or homoserine compared to non-modified microorganism.

7. The microorganism according to claim 5, wherein the microorganism belongs to the genus Corynebacterium.

8. A method for producing a target product, the method comprising culturing a microorganism in a culture medium, wherein the microorganism comprises at least any one of: a variant of an inner membrane protein YjeH comprising i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; and a polynucleotide encoding the variant.

9. The method according to claim 8, the method further comprising recovering a target product from the culture medium or the microorganism.

10. The method according to claim 8, wherein the target product is O-acetylhomoserine or homoserine.

11. A method for producing methionine, the method comprising:
producing O-acetylhomoserine by culturing a microorganism in a culture medium, the microorganism comprising at least any one of: a variant of an inner membrane protein YjeH comprising i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; and a polynucleotide encoding the variant; and
converting the O-acetylhomoserine into methionine by reacting the O-acetylhomoserine with a sulfide.

12. A method for producing glufosinate, the method comprising:
producing O-acetylhomoserine or homoserine by culturing a microorganism in a culture medium, the microorganism comprising at least any one of: a variant of an inner membrane protein YjeH comprising i) substitution of an amino acid corresponding to the 92^{nd} position with asparagine, ii) substitution of an amino acid corresponding to the 351^{st} position with leucine, or iii) substitution of both i) and ii), in an amino acid sequence of SEQ ID NO: 1, and having a homology of 95% or more and less than 100% with SEQ ID NO: 1; and a polynucleotide encoding the variant; and
converting the O-acetylhomoserine or homoserine into glufosinate.

13. A composition for producing homoserine comprising the microorganism of claim 5 or a culture of the microorganism.

14. A composition for producing O-acetylhomoserine comprising the microorganism of claim 5 or a culture of the microorganism.

15. A use of the variant of the inner membrane protein YjeH of claim 1 for producing O-acetylhomoserine or homoserine.

16. A use of the microorganism of claim 5 for producing O-acetylhomoserine or homoserine.
